SEQUENCE LISTING (13 sequences)

SEQ ID NO: 1 - protein, 14 aa, Artificial Sequence
  EIF3C peptide
QPLLLPSEDEEDTK

SEQ ID NO: 2 - protein, 28 aa, Artificial Sequence
  RPTOR peptide
GVHIHQAGGPSPPASSTSSSSLTNDVAK

SEQ ID NO: 3 - protein, 13 aa, Artificial Sequence
  AKT1S1 peptide
PSLPVSVPVWGFK

SEQ ID NO: 4 - protein, 23 aa, Artificial Sequence
  AKT1S1 peptide
RTEARSPSDEENGPPSPSPDLDR

SEQ ID NO: 5 - protein, 24 aa, Artificial Sequence
  LARP1 peptide
NALPPVLTTVNGQPSPPEHSAPAK

SEQ ID NO: 6 - protein, 26 aa, Artificial Sequence
  DPYSL2 peptide
GLYDGPVCEVSVPTPKPTVTPASSAK

SEQ ID NO: 7 - protein, 37 aa, Artificial Sequence
  DPYSL3 peptide
GMYDGPVFDLTTTPKGGPTPAGPSARGSPTRPNPPVR

SEQ ID NO: 8 - protein, 29 aa, Artificial Sequence
  MAP1A peptide
SLPSPEDAESLSVLSVPSPDTANQEPTPK

SEQ ID NO: 9 - protein, 17 aa, Artificial Sequence
  MAP1A peptide
SEPQDFQEADPSWGDTK

SEQ ID NO: 10 - protein, 28 aa, Artificial Sequence
  MAP1B peptide
VLSPLRSPPLIGSEPSAPYESFLSADDK

SEQ ID NO: 11 - protein, 33 aa, Artificial Sequence
  MAP1B peptide
TTRPTPEEGGYSYDISEKTTPSPPEVSGYSYEK

SEQ ID NO: 12 - protein, 24 aa, Artificial Sequence
  MAP2 peptide
VDHGAEIITQPSPGRSPSVAPSPR

SEQ ID NO: 13 - protein, 30 aa, Artificial Sequence
  MAP2 peptide
AGKSGTSTPPTTPGSTAITPGPTPPSYSSR

(19) United States
(12) Patent Application Publication
(10) Pub. No.: US 2024/0122939 A1
(43) Pub. Date: Apr. 18, 2024

(54) THERAPIES FOR THE TREATMENT OF DISEASES AND DISORDERS ASSOCIATED WITH ABNORMAL EXPRESSION OF CDKL5 GENE

(71) Applicant: THE REGENTS OF THE UNIVERSITY OF CALIFORNIA, Oakland, CA (US)

(72) Inventors: Alysson R. Muotri, San Diego, CA (US); Cleber Trujillo, Cambridge, MA (US)

(21) Appl. No.: 18/273,491

(22) PCT Filed: Feb. 25, 2022
(86) PCT No.: PCT/US2022/017851

§ 371 Date: Jul. 20, 2023

Related U.S. Application Data
(60) Provisional application No. 63/154,566, filed on Feb. 26, 2021.

(51) Int. Cl. A61K 31/55; A61K 31/4725; A61K 31/5377; A61P 25/00; G01N 33/50

(52) U.S. Cl. CPC A61K 31/55; A61K 31/4725; A61K 31/5377; A61P 25/00; G01N 33/5058; G01N 2500/10

(57) ABSTRACT

The disclosure provides methods for rescuing defects caused by abnormal CDKL5 expression in a subject in need thereof, comprising administering to the subject therapeutically effective amount(s) of a hyperpolarization-activated cyclic nucleotide-gated (HCN) channel blocker, a muscarinic receptor inhibitor, a GSK3 inhibitor, a Notch inhibitor and any combination thereof. The disclosure further provides methods for screening candidate drug candidates in a tiered series of assays and models (neurons, CDKL5-mosaic neurospheres, and cortical organoids).

### CROSS REFERENCE TO RELATED APPLICATIONS

This application claims priority under 35 U.S.C. § 119 from Provisional Application Ser. No. 63/154,566, filed Feb. 26, 2021, the disclosures of which are incorporated herein by reference.

### TECHNICAL FIELD

The disclosure provides for the discovery and development of therapies that can rescue neuronal defects caused by abnormal expression of neural-associated gene.

### BACKGROUND

Epilepsy is a common neurological disorder in early childhood, leading to a high burden of cognitive and behavioral comorbidity. Seizures reflect a transient, abnormal and synchronous hyperactivity of a neuronal population due to an imbalance between inhibitory and excitatory neurotransmission, resulting in tonic depolarizations or rhythmic burst discharges. Therefore, epilepsy is not a singular disease entity, but a multifactor and diverse symptom condition that may result from different causes ranging from inherited mutations to structural brain abnormalities. In this context, single-gene epilepsies are mainly composed of mutations in the PRRT2, SCN1A, KCNQ2, SLC2A1 or CDKL5 genes.

### SUMMARY

The disclosure provides drug screening platforms and treatments for CDKL5 deficiencies, including Rett Syndrome, CDD (and orphan indication). The disclosure provides a method of screening and treating CDKL5 disorders. Useful drugs that were identified and can be used for treatment include Ivabradine, Solifenacin, Crenigacestat, and AZD1080.

The disclosure provides a method for treating a disease or disorder caused by abnormal CDKL5 expression in a subject in need thereof, comprising administering to the subject a therapeutically effective amount(s) of an agent selected from the group consisting of Ivabradine, Solifenacin, AZD1080, Crenigacestat and any combination thereof. In one embodiment, the method comprises administering a single pharmaceutical composition formulated for oral delivery comprises the Ivabradine, Solifenacin, AZD1080, Crenigacestat or any combinations thereof. In another embodiment, the abnormal CDKL5 expression is cause by a genetic disease or disorder that affects the expression of the CDKL5 gene or the activity of the CDKL5 protein. In a further embodiment, the genetic disease or disorder is CDKL5 deficiency disorder (CDD). In another embodiment, the subject is a female subject. In another embodiment, the subject is a male subject. In another embodiment, the subject is less than 25 years of age. In a further embodiment, the subject is less than 10 years of age.

The disclosure also provide a method of treating CDKL5 deficiency disorder (CDD) in a subject, comprising administering to the subject a therapeutically effective amount(s) of an agent selected from the group consisting of Ivabradine, Solifenacin, AZD1080, Crenigacestat and any combination thereof. In one embodiment, the method comprises administering a single pharmaceutical composition formulated for oral delivery comprises the Ivabradine, Solifenacin, AZD1080, Crenigacestat or any combinations thereof. In another embodiment, the CDD is the result of abnormal CDKL5 expression. In a further embodiment, the abnormal CDKL5 expression is cause by a genetic disease or disorder that affects the expression of the CDKL5 gene or the activity of the CDKL5 protein. In another embodiment, the subject is a female subject. In still another embodiment, the subject is a male subject. In another embodiment, the subject is less than 25 years of age. In a further embodiment, the subject is less than 10 years of age.

The disclosure also provides a pharmaceutical composition comprising at least 2 agents selected from the group consisting of Ivabradine, Solifenacin, AZD1080, Crenigacestat and salts of any of the foregoing.

The disclosure also provides a human-based neural drug screening platform for identifying therapeutic compounds that can ameliorate or rescue deleterious biological effect(s) resulting from abnormal expression or activity of CDKL5, comprising: (a) contacting set(s) of neurons, that have been differentiated from human stem cells, with a candidate drug, wherein a first set of neurons have been differentiated from human pluripotent stem cells that have mutation(s) affecting the normal expression of a CDKL5 gene and optionally, a second set of neurons that are differentiated from pluripotent stem cells that do not have said mutation(s); and/or (b) contacting set(s) of neurospheres, that have been generated from human stem cells, with the candidate drug, wherein a first set of neurospheres comprises neurospheres that have been differentiated from human pluripotent stem cells that have mutation(s) affecting the normal expression of a CDKL5 gene, and optionally, a second set of neurospheres that have been differentiated from the human pluripotent stem cells that do not have said mutation(s); and/or (c) contacting set(s) of cortical organoids, that have been generated from human stem cells, with the candidate drug, wherein a first set of cortical organoids have been differentiated from human pluripotent stem cells that have mutation(s) affecting the normal expression of a CDKL5 gene, and optionally, a second set of cortical organoids that have been differentiated from the human pluripotent stem cells that do not have said mutation(s); (d) evaluating whether the candidate drug rescues or ameliorates deleterious biological effect(s) resulting from abnormal expression of a CDKL5 gene in the first set of neurons, and/or the first set of neurospheres, and/or the first set of cortical organoids, wherein if a candidate drug rescues and/or ameliorates one or more deleterious biological effects the candidate drug is a therapeutic compound. In another embodiment, the platform comprises neurons, neurospheres and/or cortical organoids that have been produced from induced pluripotent stems cells. In another embodiment, the induced pluripotent stem cells are dedifferentiated from cells isolated from a subject having a CDKL5 deficiency disorder (CDD).

The disclosure also provides a culture of neuronal cells, neurospheres and/or cortical organoids differentiated from stem cells obtained or induced from a subject having a CDKL5 deficiency disorder.

### DESCRIPTION OF DRAWINGS

FIG. 1A-H shows altered proliferation and viability in CDD progenitor cells. (A) Schematic representation of the CDKL5 gene showing the mutations exhibited by CDD cell lines used in the study. (B) Left, each cell line is represented by an individual-colored symbol that is used throughout the study. CDD and related controls share the same symbol; filled symbols represent isogenic cells. Right, overview of the assays performed using neural progenitor cells (NPCs). Representative images showing stage-specific protein expression in iPSCs and NPCs. Scale bar, 100 μm. (C) Volcano plot illustrating the global proteomic (top) and phosphoproteomic (bottom) changes in CDD vs control NPCs. The y-axis shows the −log 10 of the t-test P value. A P value of 0.05 and a |log 2 (fold change)| of 0.5 are indicated by dotted lines (Control, n=4 cell lines; CDD, n=4 cell lines; each cell line was derived from a different subject). (D) ClueGO analysis of differentially expressed proteins in CDD NPCs. The size of the circles indicates its significance, and similar terms are grouped with the same color (Control, n=4 cell lines; CDD, n=4 cell lines). (E) Ingenuity Pathway Analysis (IPA) showing differentially expressed proteins (DEPs) in the NPC dataset and the predicted effect of these changes. Top, DEPs involved in cell cycle progression. Bottom, predicted effects on development of neurons, and cell viability. (Control, n=4 cell lines; CDD, n=4 cell lines). (F) Representative phase-contrast images showing the differences in confluency between CDD and control NPCs at day 2 and, ratio of CDD NPC number at day 2 over day 0 relative to control. Scale bar, 100 μm (Control, n=9, 3 cell lines; CDD, n=9, 3 cell lines; minimum of two technical replicates per cell line; two-tailed Mann-Whitney U test, ****P<0.0001). (G) Percentage of cells at different phases of the cell cycle compared to control (Control, n=16, 4 cell lines; CDD, n=16, 4 cell lines; four technical replicates per cell line; two-way ANOVA, *P<0.05). (H) Cell death analysis of CDD NPCs compared to control (Control, n=16, 8 and 11, respectively for fragmented DNA, caspase+ cells and depolarized mitochondria, 4 cell lines; CDD, n=16, 8 and 11, respectively for fragmented DNA, caspase+ cells and depolarized mitochondria, 4 cell lines; minimum of two technical replicates per cell line; two-tailed Mann-Whitney U test, **P=0.0110, ****P<0.0001). In all assays, each cell line was derived from a different subject. In (F)-(H), data are shown as mean±s.e.m.; individual values are indicated by dots where each symbol represents a subject.

FIG. 2A-I shows proteomics and phosphoproteomics analysis of mTOR alterations in CDD neural cells. (A) Schematic diagram showing the iPSC differentiation into neural lineages, and summarizing the assays performed with each cell type. Representative phase-contrast images of NPCs, neurons and cortical organoids are shown. Scale bar, 100 μm. (B) Stage-specific protein expression in 6-week-old cortical neural cultures. Scale bar, 100 μm. (C) ClueGO analysis of differentially expressed phosphoproteins in 6-week-old CDD neurons (Control, n=5 cell lines; CDD, n=5 cell lines). (D) ClueGO analysis of differentially expressed phosphoproteins in 2-month-old CDD cortical organoids (Control, n=4 cell lines; CDD, n=4 cell lines). The size of the circles indicates its significance, and similar terms are grouped as the same color. (E) Top enriched terms for significantly regulated phosphoproteins in the category of nervous system development and function in Ingenuity Pathway Analysis (IPA). (F) Relative phosphorylation of proteins at downstream of mTOR signaling pathway in 6-week-old neurons. Relative ratios of phosphopeptide intensity normalized to non-phosphoprotein intensity are shown (Control, n=5 cell lines; CDD, n=5 cell lines; unpaired t-test, each row was analyzed individually without assuming a consistent SD, *P<0.05, **P<0.01, ***P<0.001); SEQ ID Nos:1-5 in order of appearance. (G) and (H) Quantification of phosphorylated-S6/total S6 ratio from Western blot using protein lysates from 6-week-old CDD and control neurons (Control, n=6 cell lines; CDD, n=6 cell lines; two-tailed Mann-Whitney U test, **P=0.0087). The band intensity was quantified using a LI-COR imaging system and the P-S6 to S6 ratio was calculated for each sample. (I) Time-dependent responses of CDD and control cells upon amino acid (aa) starvation. CDD and control NPCs were incubated with media containing or lacking amino acids for the indicated times. mTORC1 activity was determined by Western blot with antibodies against phospho-p70S6K, phospho-S6, phospho-4E-BP1 and total p70S6K, S6 and 4E-BP1 (Control, n=3 cell lines; CDD, n=3 cell lines). In (F) and (I) data are shown as mean±s.e.m. Individual values are indicated by dots where each symbol represents a subject. CDD and related controls share the same symbol and filled symbols represent isogenic cells.

FIG. 3A-G shows cellular and morphological defects in CDD neurons. (A) Representative images of neuronal spine-like structures in 8-week-old CDD and control neurons transduced with Synapsin1 promoter-driven EGFP expression. The time series images were taken every 5 min. Scale bar, 10 μm (Motility: Control, n=28, 3 cell lines; CDD, n=30, 3 cell lines; Density: Control, n=22, 5 cell lines; CDD, n=17, 3 cell lines; minimum of four technical replicates per cell line; two-tailed Mann-Whitney U test, *P<0.05, ****P<0.0001). (B) Left, representative immunostaining images of EGFP+ and CTIP2+ neurons used for neuronal tracing. CTIP2 was used as cortical layers V/VI marker. Right, morphometric analyses showing significant differences in total dendritic length and dendritic segment number, but not in cell body area of 8-week-old CDD neurons compared to controls. Scale bar, 20 μm (Control, n=64, 5 cell lines; CDD, n=64, 5 cell lines; minimum of three technical replicates per cell line; two-tailed Mann-Whitney U test, ***P=0.0004 for dendritic length and, ***P=0.0002 for dendritic segment number). (C) Representative images of tracings from CDD and control neurons in vitro. Graph showing Sholl analysis of the dendritic complexity of EGFP+ neurons. Number of intersections refers to the number of dendrites intersecting concentric circles spaced 10 μm apart starting from the cell body (Control, n=12, 5 cell lines; CDD, n=15, 5 cell lines; unpaired t-test, each row was analyzed individually without assuming a consistent SD, *P<0.05, **P<0.01). (D) Left, schematic of the chimeric human-mouse brain approach. Middle, morphometric analysis showing significant difference in total dendritic length between CDD and control neurons at 6 months after transplantation. Right, Sholl analysis of the dendritic complexity of EGFP+ human neurons in vivo (Control, n=10, 2 cell lines; CDD, n=12, 2 cell lines; Dendritic length: two-tailed Mann-Whitney U test, *P<0.0358; Sholl analysis: unpaired t-test, each row was analyzed individually without assuming a consistent SD, *P<0.05). (E) Representative images of post-mortem cortical layer V/VI pyramidal neurons using Golgi staining. Scale bar, 25 μm. (F) Neuronal tracing of post-mortem neurons from CDD and control. The tissue was collected at 5- and 6-year-old, respectively for CDD and control subjects. Left, representative images of tracings. Right, Sholl analysis of the dendritic complexity of silver stained neurons. Number of intersections refers to the number of dendrites intersecting concentric circles spaced 20 μm apart starting from the cell body. Scale bar, 20 μm (Control, n=10 neurons, 1 subject; CDD, n=10 neurons, 1 subject; unpaired t-test, *P<0.05, **P<0.01). (G) Morphometric analyses of post-mortem cortical neurons showing differences in total dendritic length and cell body area between CDD and control (Dendritic length: Control, n=17 neurons, 1 subject; CDD, n=17 neurons, 1 subject; Cell body area: Control, n=40 neurons, 1 subject; CDD, n=40 neurons, 1 subject; two-tailed Mann-Whitney U test, **P<0.01, ****P<0.0001). Data are shown as mean±s.e.m. Individual values are indicated by dots where each symbol represents a subject. CDD and related controls share the same symbol and filled symbols represent isogenic cells.

FIG. 4A-K shows altered synaptic and network activity in CDD neurons. (A) Expression levels of pre- and post-synaptic markers (respectively, Synapsin1 and PSD95) in 8-week-old CDD neurons compared to control, assessed by Western blot analysis (Control, n=6 cell lines; CDD, n=6 cell lines; two-tailed Mann-Whitney U test, *P=0.0260, **P=0.0043). (B) Representative images (left) and puncta quantification (right) of MAP2+ neurons stained for pre- and post-synaptic markers (VGLUT1 and HOMER1, respectively) (Control, n=83, 5 cell lines; CDD, n=108, 5 cell lines; minimum of 13 technical replicates per cell line, two independent experiments; two-tailed Mann-Whitney U test, ****P<0.0001). Scale bar, 5 μm. (C) Heatmap of all interactions significantly different between CDD and control cortical organoids by ANC∩CNA. Rows indicate interactions, columns indicate experimental replicates. Data are clustered by column and scaled by row to represent intensity values that vary over 3 orders of magnitude (Control, n=8, 2 cell lines; CDD, n=8, 2 cell lines; four technical replicates per cell line, two independent experiments). (D) Node-edge representation of interactions listed in (C). Edge color and thickness indicates the magnitude of the log 2 fold change between CDD and control organoids. (Control, n=8, 2 cell lines; CDD, n=8, 2 cell lines; four technical replicates per cell line, two independent experiments). (E) Whole-cell patch clamping of differentiated neurons spread in the dish. Left, representative image of patch-clamp neurons. Scale bar, 10 μm. Right, resting membrane potential (RMP) and rheobase currents in CDD neurons compared to control (RMP: Control, n=19, 2 cell lines; CDD, n=42, 3 cell lines; Rheobase: Control, n=15, 2 cell lines; CDD, n=35, 3 cell lines; minimum of 6 technical replicates per cell line; two-tailed Mann-Whitney U test, *P=0.0386, **P=0.0037). (F) Left, representative action potential (AP) firing traces evoked from −60 mV in CDD and control neurons. Right, number of evoked APs in response to current steps (0-50 pA) from resting membrane potential (Control, n=17 neurons, 2 cell lines; CDD, n=28 neurons, 3 cell lines; minimum of 4 technical replicates per cell line; unpaired t-test, each row was analyzed individually without assuming a consistent SD, *P<0.05, **P<0.01). (G) Left, representative recordings of spontaneous excitatory synaptic currents of CDD and control neurons voltage-clamped at −60 mV. Right, sEPSC frequency observed in CDD neurons compared to control (Control, n=17, 2 cell lines; CDD, n=22, 3 cell lines; minimum of 5 technical replicates per cell line; two-tailed Mann-Whitney U test, ****P<0.0001). (H) Schematic of cortical organoid electrophysiological recording using a multi-electrode array (MEA). (I) Representative raster plots of CDD and control cortical organoid neural network activity at 16 weeks of differentiation. (J) and (K) Functional characterization of CDD cortical organoids showing changes in the mean firing rate (J) and synchrony index (K) over time, compared to control (Control, n=6, 2 cell lines; CDD, n=6, 2 cell lines; 3 technical replicates per cell line; unpaired t-test, *P=0.0111, **P=0.0030). Data are shown as mean±s.e.m. Individual values are indicated by dots where each symbol represents a subject. CDD and related controls share the same symbol and filled symbols represent isogenic cells.

FIG. 5A-M presents high throughput screening and functional rescue of CDD neural network. (A) High throughput (HT) format for CDD phenotypic screening in 384-well plates. Spheroids comprise a defined culture of cortical neurons and astrocytes. (B) Functional characterization of CDD and control neural networks over time. Each peak represents a spontaneous calcium oscillation that correlates with synchronous neural activity. (C) Changes in peak amplitude and peak frequency in control and CDD culture over time (2-, 3-, and 4-weeks, n=32 spheroids for control and CDD; 6 weeks n=96 and 256 spheroids, respectively, for control and CDD; 1 cell line; ****P<0.0001). Data are shown as mean±s.d. (D) Schematic of the HT screening protocol. (E) Z-score of 17 calcium oscillation features in CDD neural networks compared to control at 6 weeks of differentiation. DMSO (0.001%, top) and water (bottom) were used as vehicle for the screening compounds. (F) Area of control, CDD-untreated and CDD-treated spheroids after 6 weeks of differentiation. Chronic treatment with 1112 compounds was performed for three weeks (Control, n=48, 1 cell line; CDD-untreated, n=88, 1 cell line, CDD-treated, n=1 spheroid per compound). Data are shown as mean±s.d. (G) Cell viability of control (black), CDD-untreated (gray) and CDD-treated (blue) spheroids after 6 weeks of differentiation. Chronic treatment with 1112 compounds was performed for three weeks (Control, n=96, 1 cell line; CDD-untreated, n=176, 1 cell line, CDD-treated, n=2 spheroids per compound). Data are shown as mean±s.d. (H) Radar plot showing the functional calcium oscillation phenotype signature of CDD-untreated (gray line) compared to control (black line) spheroids. (I) and (J) Distribution of CDD-treated calcium activity according to the Scalar Perturbation (I) and Recovery (J) parameters. In blue, top 50 compounds with lowest values for Scalar Perturbation and, top 25 compounds with more than 60% rescue for Parameter Recovery. (K) Radar plots showing the functional rescue of CDD calcium oscillation signature after treatment with selected compounds. (L) Representative calcium oscillation tracings from spheroids control, CDD-untreated and CDD-treated with selected compounds. (M) Dose-response curve considering the Parameter Recovery percentage for selected compounds (n=4 spheroids per concentration for each treatment, 1 cell line). Data are shown as mean±s.e.m.

FIG. 6A-G shows the characterization of CDD and control iPSC lines. (A) Information of the subjects included in this study. (B) Representative immunofluorescence images of markers for pluripotency in CDD and control iPSCs. Scale bar, 100 μm. (C) Expression of pluripotency gene markers in fibroblasts, ESC and iPSCs. In the Euclidian matrix distance-based heatmap and hierarchical clustering-based dendrogram (left), the variability of expression among samples is indicated by Z-score. (D) Representative images of male and female karyotypes of CDD cell lines assessed by G-banding analysis. (E) Representative graphical outputs of SNP-based arrays for copy number variation analysis in male and female iPSC lines. (F) Representative images of iPSCs immunostained with antibodies for H3K27me3. The single punctate foci of H3K27me3 in female cells (top) indicates the presence of an X chromosome inactivation (Xi), while no apparent nuclear accumulation of H3K27me3 is seen in male cells (bottom). (G) RNA sequence chromatogram showing the CDKL5 nucleotide changes in CDD lines compared to controls.

FIG. 7A-L shows proteomic and phosphoproteomic characterization of CDD neural cell lines. (A) and (B) Gene expression levels of CDKL5 (A) and CDKL5 protein content (B) in control cells at different stages of differentiation (iPSC, NPC and 6-week-old neurons). (C) CDKL5 protein content observed in 6-week-old neurons derived from CDD patients and control individuals, compared to post-mortem human cortical brain lysate. (D) No differences are observed in the nucleus area and number of MAP2+ cells of 2-month-old CDD organoids compared to control. (E) Volcano plots illustrating the global proteomic and phosphoproteomic changes in 2-month-old CDD organoids compared to control. All the quantified proteins were plotted (Control, n=4 cell lines; CDD, n=4 cell lines). (F) Biological functions and diseases showing a significant overlap with phosphorylation changes in 6-week-old CDD neurons (Control, n=5 cell lines; CDD, n=5 cell lines). (G) Relative phosphorylation of representative microtubule associated proteins in 6-week-old neurons. Relative ratios of phosphopeptide intensity normalized to non-phosphoprotein intensity are shown (Control, n=5 cell lines; CDD, n=5 cell lines; unpaired t-test, *P<0.05, **P<0.01, ***P<0.001). (SEQ ID NO:6-13 in order of appearance). (H) Site-specific phosphorylation changes in proteins related with microtubule dynamics in CDD neurons (Control, n=5 cell lines; CDD, n=5 cell lines). (I) TMT reporter ion intensity (normalized to total intensity of each channel) showing that GABA receptor proteins are downregulated in 6-week-old CDD neurons (Control, n=5 cell lines; CDD, n=5 cell lines). (J) Phosphorylation changes associated with mTOR pathway in CDD neurons. Phosphoproteins increased in CDD neurons are highlighted (diagram adopted from IPA). (K) Western blot quantification of proteins at downstream of mTOR signaling pathway in CDD and control neurons lysates (Control, n=6 cell lines; CDD, n=6 cell lines; two-tailed Mann-Whitney U test). Data are shown as mean±s.e.m. Individual values are indicated by dots where each symbol represents a subject. CDD and related controls share the same symbol and filled symbols represent isogenic cells. (L) Time-dependent responses of CDD and control NPCs upon amino acid starvation. The Western blot band intensity was quantified using a LI-COR imaging system and the phosphorylated/total ratio was calculated for each target considering each CDD sample and its related control.

FIG. 8A-H shows morphometric analysis of CDD neurons. (A) CDD neurons show no differences in the number of dendrites or branching points compared to control in vitro (Control, n=64 and 14, respectively for number of dendrites and branching points, 5 cell lines each; CDD, n=64 and 13, respectively for number of dendrites and branching points, 5 cell lines each; minimum of three technical replicates per cell line; two-tailed Mann-Whitney U test). (B) Top, Representative immunofluorescence images of human neurons transplanted into the mouse brain. Scale bar, 100 μm. Middle, the spine density and complexity of human neurons transplanted into the mouse brain increase over time. Bottom, Mouse brain sections with HuNu-positive cells from anterior to posterior. Scale bar, 1000 μm. Representative immunofluorescent images of transplanted human neurons after 6-month differentiation. (C) No significant differences were observed in cell body area, number of dendrites, branching points or dendritic segment between CDD and control neurons at 6 months after transplantation into a mouse brain (Control, n=10, 2 cell lines; CDD, n=12, 2 cell lines; minimum of five technical replicates per cell line). (D) Representative images of post-mortem cortical layer V/VI neurons from CDD and control subjects using Golgi staining. Scale, 25 μm. (E) Morphometric analyses of post-mortem cortical layer V/VI pyramidal neurons from different Broadman areas (BA). Post-mortem tissues obtained from CDD and control subjects at 5- and 6-year-old, respectively. (F) Morphometric analyses of post-mortem cortical layer V/VI pyramidal neurons obtained from 30-year-old subjects. Note the differences in cell body area between CDD and control depending on the BA under evaluation. (G) Left, representative contrast-phase images of cortical organoids at different stages of development. Scale bar, 200 μm. Right, diameter measurements performed at the maturation stage (Control, n=165, 2 cell lines; CDD, n=217, 2 cell lines; minimum of seventy technical replicates per cell line; two-tailed Mann-Whitney U test, ****P<0.0001). (H) Left, representative contrast-phase images of neural cell migration from cortical organoids plated at early stages of development. Scale bar, 200 μm. Right, migration distances measured five days after plating (Control, n=25, 3 cell lines; CDD, n=20, 3 cell lines; minimum of four technical replicates per cell line; two-tailed Mann-Whitney U test, ****P<0.0001). Data are shown as mean±s.e.m. Individual values are indicated by dots where each symbol represents a subject. CDD and related controls share the same symbol and filled symbols represent isogenic cells.

FIG. 9A-F shows synaptogenesis and electrophysiological activity of CDD neurons. (A) Western blot showing the expression levels of pre- and post-synaptic proteins (Synapsin 1 and PSD-95, respectively) in 8-week-old CDD neurons and controls. (Control, n=6 cell lines; CDD, n=6 cell lines; each cell line was derived from a different subject). (B) Principal component plot of protein interaction network matrices showing two technical replicates run in parallel (connected by lines) from each of two CDD and control (CTL) lines. The QMI experiment was repeated twice (R1 and R2) to illustrate batch and background effects (Control, n=8, 2 cell lines; CDD, n=8, 2 cell lines; four technical replicates per cell line, two independent experiments). (C) No differences were observed in cell capacitance (Cm), action potential (AP) amplitude or width in CDD neurons compared to control by whole-cell patch clamping (Cm: Control, n=20, 2 cell lines; CDD, n=48, 3 cell lines; AP amplitude: Control, n=14, 2 cell lines; CDD, n=39, 3 cell lines; AP 4 width: Control, n=14, 2 cell lines; CDD, n=29, 3 cell lines; minimum of four technical replicates per cell line; two-tailed Mann-Whitney U test). (D) Current densities of and I_{KA} (left) and Na current densities (right) for CDD and control neurons (**P<0.01 and ****P<0.0001). (E) Spontaneous excitatory postsynaptic current (sEPSCs) amplitude in CDD and control neurons (****P<0.0001). Data are shown as mean±s.e.m.; individual values are indicated by dots where each symbol represents a subject; CDD and related controls share the same symbol; filled symbols represent isogenic cells. (F) Network activity of CDD and control cortical organoids over time, characterized by different parameters using the MEA (Control, n=6, 2 cell lines; CDD, n=6, 2 cell lines; three technical replicates per line; unpaired t-test, each row was analyzed individually without assuming a consistent SD, *P<0.05). Data are shown as mean±s.e.m.

FIG. 10A-H shows characterization of CDD high throughput screening platform. (A) and (B) Box plot showing the differences in peak count (A) and amplitude (B) for CDD and control calcium oscillation activity in spheroids. DMSO (0.001%) and water were used as vehicle for the screening compounds (Control, n=72, 1 cell line; CDD, n=408, 1 cell line). Data are shown as mean±s.d. (C) and (D) Principal component analysis (PCA) was used to compare the distribution of CDD and control calcium oscillation activity on the basis of peak count and amplitude. DMSO (0.001%) (C) and water (D) were used as vehicle for the screening compounds (Control, n=72, 1 cell line; CDD, n=408, 1 cell line). (E) Changes in the peak classification of CDD and control calcium oscillations over time. (F) Representative contrast-phase images of CDD and control spheroids over time. (G) Area of control and CDD-untreated spheroids over time (Control, n=48, 1 cell line; CDD-untreated, n=88, 1 cell line). Data are shown as mean±s.d. (H) Top enriched terms related to the top 50 compounds able to rescue more than 60% of CDD calcium oscillation activity; one-way ANOVA analysis, ****P<0.0001). Data are shown as mean±s.e.m.

### DETAILED DESCRIPTION

As used herein and in the appended claims, the singular forms “a,” “an,” and “the” include plural referents unless the context clearly dictates otherwise. Thus, for example, reference to “a prodrug” includes a plurality of such prodrugs and reference to “the agent” includes reference to one or more agents and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this invention is not limited to the particular methodology, protocols, and reagents, etc., described herein and as such may vary. The terminology used herein is for the purpose of describing particular embodiments only and is not intended to limit the scope of the invention, which is defined solely by the claims.

Other than in the operating examples, or where otherwise indicated, all numbers expressing quantities of ingredients or reaction conditions used herein should be understood as modified in all instances by the term “about.” The term “about” when used to described the invention, in connection with percentages means±10%, 5%, or typically 1%.

The human CDKL5 gene encodes for a protein with a highly conserved serine-threonine kinase domain in its N-terminal, sharing homology to both members of the mitogen-activated protein (MAP) kinase and cyclin-dependent kinase (CDK) families. CDD is one of the lead causes of genetic early-onset epilepsy and is characterized as a devastating neurodevelopmental disorder that has no cure and still lacks effective treatments. Much effort has been made in correlating genotype to symptoms, but little is known about its pathophysiology, mainly due to scarce information on CDKL5 protein function and downstream targets. Notably, mouse models for CDD have been controversial regarding the emergence of spontaneous seizures to mirror the human condition. Therefore, the early-onset intractable seizures and the lack of robust mouse models, make the use of iPSC from CDD patients a valid approach to understanding its molecular mechanism and screen novel lead compounds for more effective treatments.

The cyclin-dependent kinase-like 5 (CDKL5) gene localizes on chromosome Xp22 and encodes for a serine/threonine kinase highly expressed in the central nervous system. Mutations in this gene cause CDKL5 deficiency disorder (CDD), characterized by neurodevelopmental delay, motor dysfunction, autistic features and early-onset intractable seizures, a defining trait that led to the standalone classification of this pathology. Due to CDKL5's X-linkage, female heterozygous CDD patients show a spectrum of phenotypes based on the mosaicism generated by X-chromosome random inactivation. Hemizygous males, with a nonfunctional copy of the gene, likely display more severe clinical symptoms. Nonetheless, both male and female CDD patients exhibit various mutations (translocations, nonsense, missense, frameshift and splice variants), mostly found in the N-terminal kinase domain, resulting in an absent or malfunctioning CDKL5 protein.

CDKL5 is crucial for proper brain development and neuronal function, but its precise targets in relevant cell types are still yet to be determined. To clarify the effects of CDKL5 loss-of-function and the etiology of CDD, several mouse models have been generated. Specific behavioral abnormalities such as cognitive deficits and impaired motor control have been observed in these models. However, none of the rodent models showed a consistent and robust recapitulation of the human condition, including the absence of spontaneous seizures in early development. These phenotypes were accompanied by neuroanatomical variations, such as altered dendritic arborization, spine defects, and compromised neuronal connectivity, along with disruption of signaling pathways.

Epilepsy is one of the most common neurological disorders, categorized mainly by atypical neuronal network electrical activity, leading to recurrent and unpredictable seizures. Treatment, which generally includes medications or sometimes surgery, may eliminate or reduce the frequency and intensity of seizures; however, the mechanism(s) underlying this condition remains unclear. Although much has been done to comprehend the development of epilepsy, the results are still insufficient for clinical translation and no effective treatment has emerged yet. In this context, by using a human neurodevelopmental experimental model, the disclosure exploits a well-defined, mono-genetically driven epilepsy phenotype. The strategic use of CDKL5-deficient cells as a biological tool to understand its complex phenotypes provides additional insight into the epilepsy field and new compounds for curative or ameliorative therapies.

The disclosure provides a human stem cell-based model for CDD by reprogramming cells from patients with different loss-of-function CDKL5 mutations that lead to an early stop codon and nonfunctional protein. In female patients, this strategy takes advantage of the random X-inactivation to identify two pairs of isogenic clones in order to minimize the influence of the individual's genetic background on the observed phenotypes. Molecular, cellular and functional analysis of cortical organoids and cells derived from patients with mutations in CDKL5 revealed its specific role in human cells. The CDD neuropathophysiology develops due to multiple alterations: the defective NPC proliferation leading to morphological changes, hyperactivation of the mTOR pathway and alterations in neuronal connectivity and excitability. In contrast to CDD rodent models showing a decrease in mTORC1 activity, the data revealed herein shows increased phosphorylation of AKT1S1, EIF3C, RPTOR, LARP1 and rpS6 in CDD neurons. Consistent with mTOR hyperactivation, CDD neuronal progenitor cells (NPCs) have slower proliferation and augmented cell death, along with sustained phosphorylation of mTOR downstream targets under amino acid starvation. The enhanced neurite outgrowth observed might be accounted for by the misregulation of chondroitin sulfate degradation observed in the proteomic CDD profile. Moreover, CDD neurons also exhibited significantly longer dendrites, increased spine-like densities, impaired spine-like motility and synaptogenesis.

The proteomics and phosphoproteomics profiles of CDD cortical neurons point to alterations in connectivity and functionality. Interestingly, the observed hyperactivation of mTOR is a hallmark of epileptic neurological disorders. Thus, the disclosure describes dynamically investigating the network activity of CDD neurons over time. CDKL5-deficient cortical organoids were significantly more active than controls, consistent with QMI data indicating increased AMPAR and mGluR levels in CDD. By evaluating the behavior of individual cells, it was confirmed that CDD neurons are more excitable than controls. This hyperexcitability of CDD cortical neurons could explain the occurrence of seizures early in the life of CDD patients.

The knowledge of new molecular signatures in CDKL5-deficient neurons prompted the development of a pharmacological strategy for targeting aberrant integrated neurons into circuits and possibly causing neuronal hyperexcitability in patients. The CDD model was implemented on a validated 3D HTS platform on an industrial scale. CDD spheroids were examined at six weeks of differentiation by high throughput calcium imaging assays. A multiparametric analysis, focusing on calcium oscillation frequency and peak irregularities, was used to rank the compound rescue effect. After ensuring that controls were distinguishable and defining a phenotypic ‘recovery’, two unbiased analysis algorithms were developed (Scalar Perturbation (SP) and Parameter Recovery (PR)) to evaluate the impact of compounds rescuing 17 activity parameters in CDD. Using these independent algorithms and considering the calcium tracing profile, spheroid area and cell viability data, top lead compounds were selected.

The alkaloid Harmine was one of the top candidate compounds ranking as first and second in the SP and PR, respectively. Notably, this compound was reported to restore CDKL5-dependent synaptic defects in Cdkl5 knockout mouse neurons. However, it was excluded from the list of top candidates due to its effect on cell survival. All the selected compounds did not significantly reduce cell viability, and some were even able to rescue the size and cellular migration defects of the spheroids.

The disclosure demonstrates the power of assessing several cellular and functional rescues before in vivo tests. This strategy can reduce downstream failures and expenses in the quest for therapeutic compounds. Using a human neurodevelopmental model, the disclosure identified potential downstream molecules and pathways affected by CDKL5 mutation in CDD brain cells and provides associated cellular and functional phenotypes related to molecular alterations, identifying therapeutic opportunities for CDD patients and other refractory epileptic syndromes.

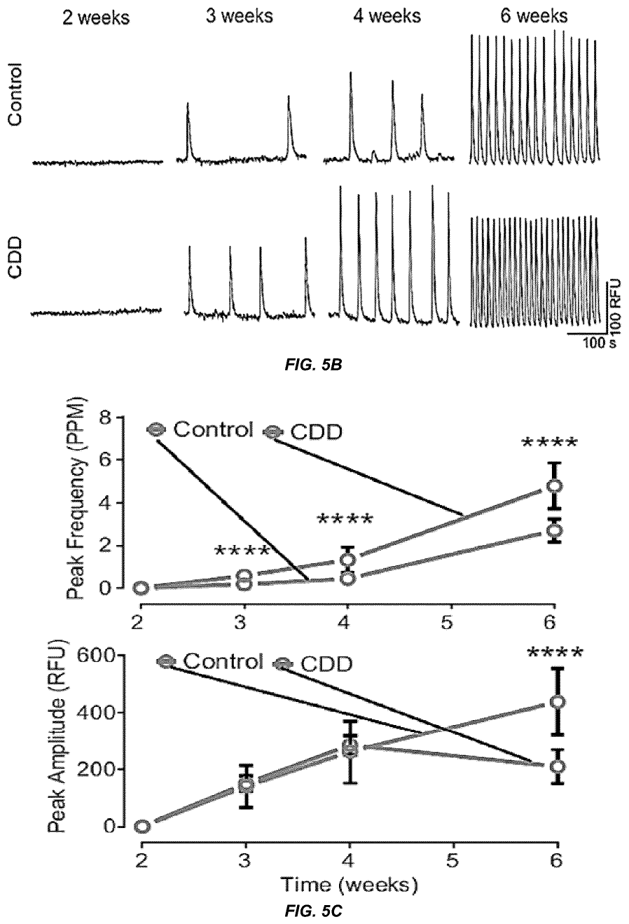

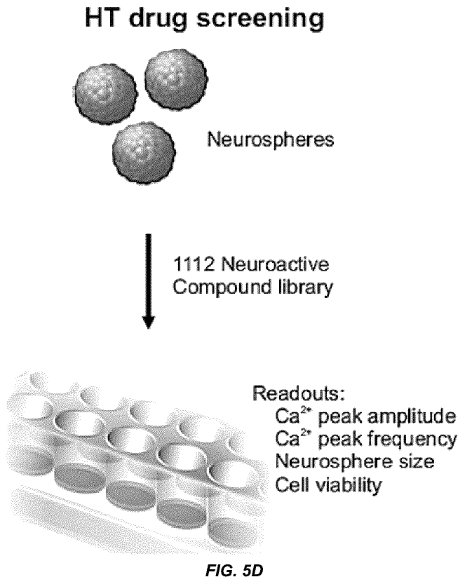

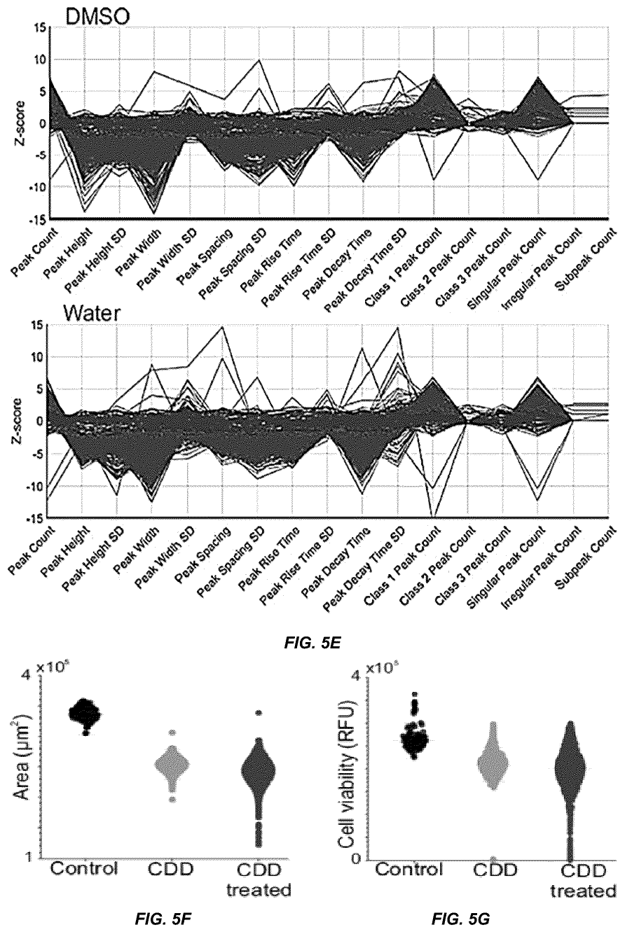

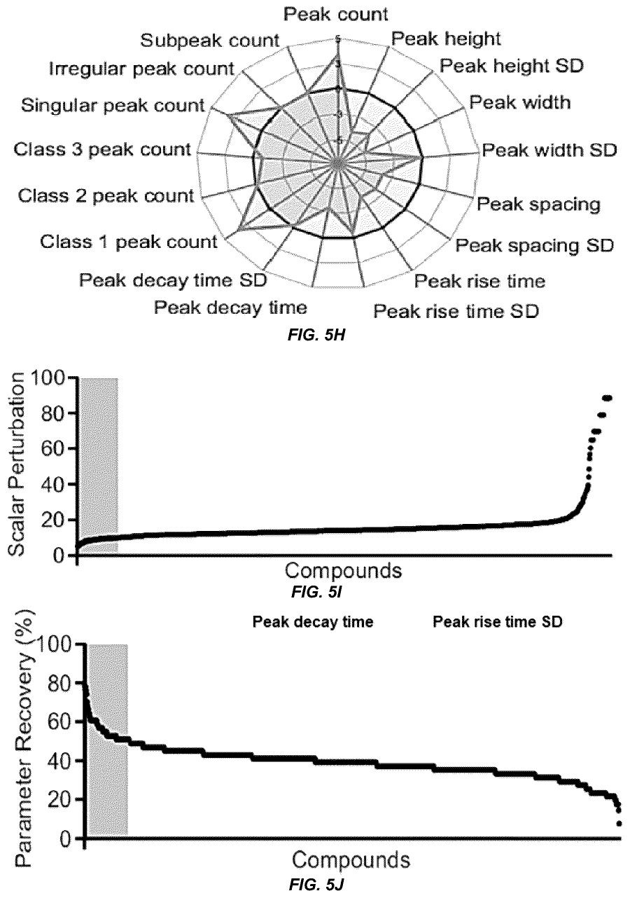

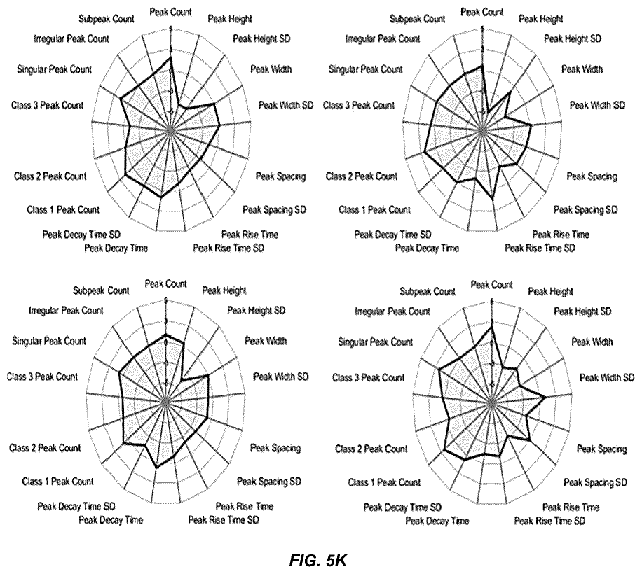

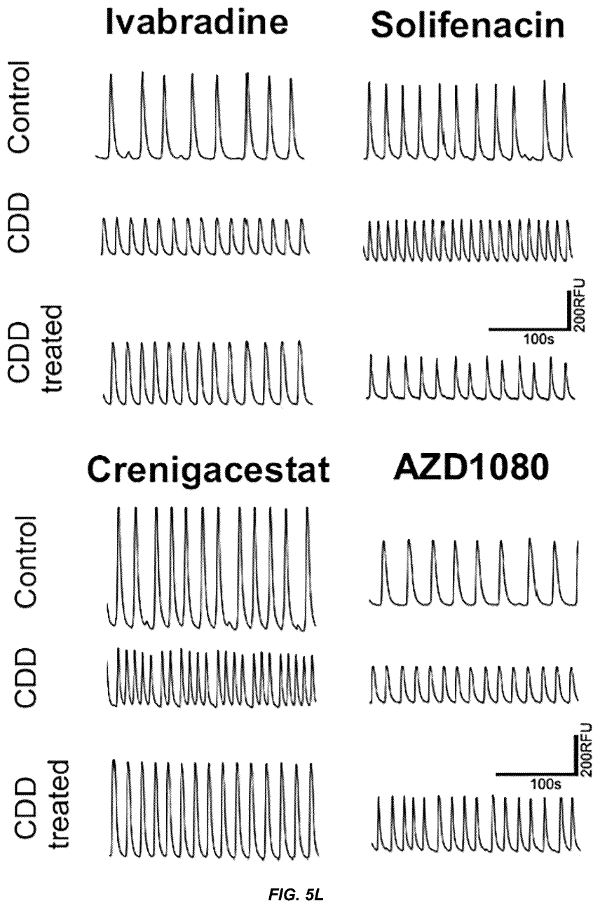

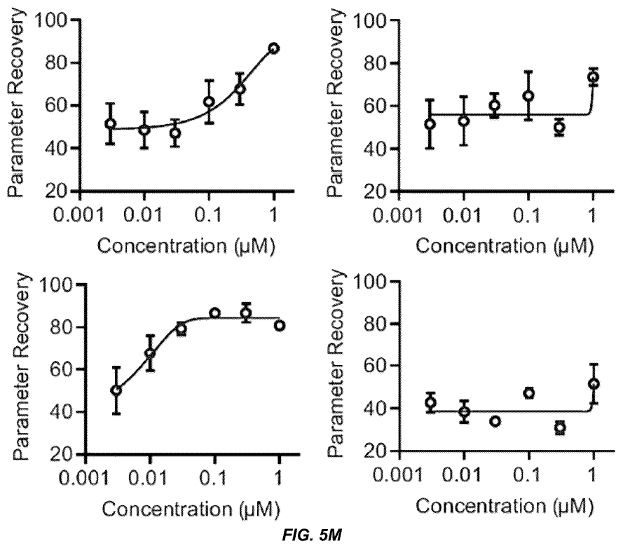

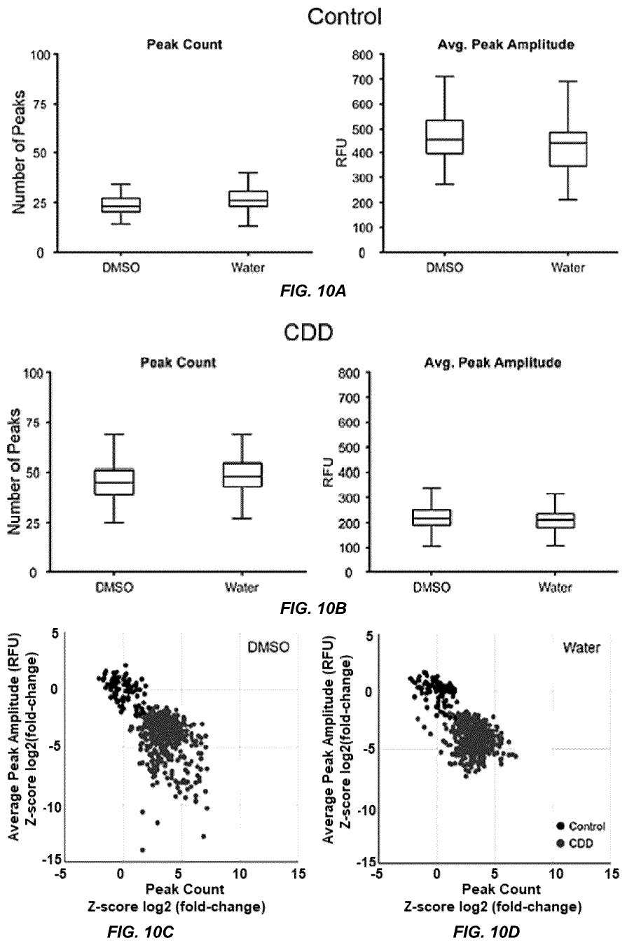

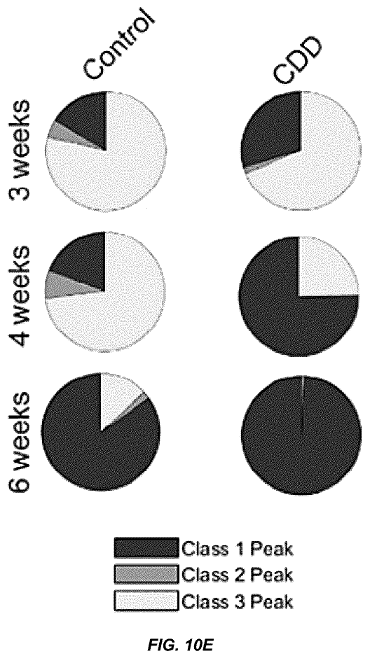

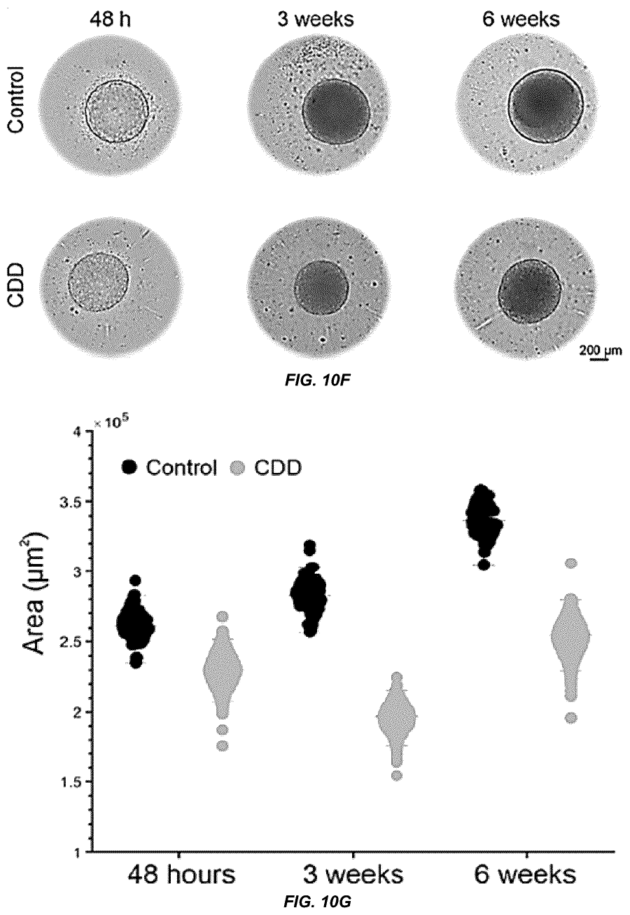

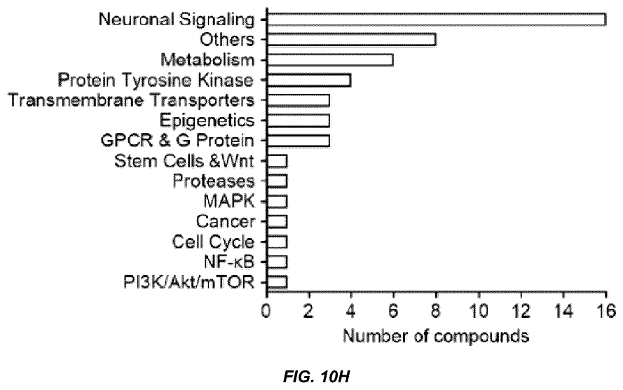

The disclosure provides a high throughput (HT) drug screening platform comprising 3D neural spheroids in a multi-well format (one spheroid per well; see, e.g., FIG. 5 and FIG. 10). This platform can be optimized to reduce variability within the genotypes, with spheroids displaying the same size across the plate, which is useful for a consistent and reproducible screening assay (FIG. 5A). Each spheroid contains a balanced population of cortical neurons and astrocytes expressing key markers of maturity and exhibits robust spontaneous, synchronous calcium oscillations. Although the spheroid genotypes showed an increase in neural activity over time, the frequency of spontaneous calcium oscillations (number of peaks) was more pronounced in CDD compared to controls, starting at early stages of the development; while the amplitude (peak height) was decreased in CDD neurons (see, e.g., FIG. 5B-C and FIG. 10E). Various readouts can be used; however, calcium oscillations was identified as a physiological readout to avoid disrupting the established 3D network5, while evaluating the spheroid size and viability.

The disclosure also provides methods for treating a subject with an X-linked neuronal disorder, or abnormal CDKL5 expression comprising administering a therapeutically effective amount of an agent selected from the group consisting of a hyperpolarization-activated cyclic nucleotide-gated (HCN) channel blocker, a muscarinic receptor inhibitor, a GSK3 inhibitor, a Notch inhibitor and any combination thereof. A therapeutically effective amount can be measured as the amount sufficient to ameliorating or abrogating symptoms associated with an X-linked disorder, or abnormal CDKL5 expression. Generally, the optimal dosage of therapeutic compound(s) will depend upon the type and stage of the neuronal disruption and factors such as the weight, sex, and condition of the subject. Nonetheless, suitable dosages can readily be determined by one skilled in the art. Typically, dosages used in vitro may provide useful guidance in the amounts useful for in situ administration of the pharmaceutical composition, and animal models may be used to determine effective dosages for treatment of specific neuronal disorders. Various considerations are described, e.g., in Langer, Science, 249:1527, (1990); Gilman et al. (eds.) (1990), each of which is herein incorporated by reference. Typically, a suitable dosage for treating an X-linked disorder, or abnormal CDKL5 expression with the therapeutic compound(s) is from 1 mg/kg to 1000 mg/kg body weight, e.g., 150 to 500 mg/kg body weight. In a particular embodiment, a therapeutic compound disclosed herein is administered at dosage of 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg, or a range that includes or is between any two of the foregoing dosages, including fractional dosages thereof.

In one embodiment, the hyperpolarization-activated cyclic nucleotide-gated (HCN) channel blocker is Ivabradine; the muscarinic receptor inhibitor is Solifenacin; the GSK3 inhibitor is AZD1080, and the Notch inhibitor is Crenigacestat.

Any reference to a drug herein also encompasses all of the pharmaceutically acceptable isomers (e.g., stereoisomers), solvates, hydrates, polymorphs, salts, and prodrugs (e.g., esters and phosphates). For example, a reference to solifenacin herein also includes its pharmaceutically acceptable salts, such as a succinate salt of solifenacin.

Further provided herein, are studies which employ an human-based model of cellular defects. The model has utility for CDKL5 and other X-linked genetic disorders, and the concept can likewise be expanded to include other aspects of pharmacogenomic precision medicine, such as characterizing pharmacologic response or variation in cytotoxicity for patients with novel mutations. Furthermore, the impact of somatic mosaicism on clinical and neurodevelopmental phenotypes is becoming increasingly appreciated, so the model developed herein will offer further utility for the study of somatic neuronal mosaicism in other neuropsychiatric diseases and human neurodevelopment in general.

The disclosure further also provides a neural drug screening platform for identifying therapeutic compounds that can ameliorate or rescue deleterious biological effect(s) resulting from abnormal expression of a gene from a chromosome (e.g., X chromosome), comprising: (a) contacting set(s) of neuronal cells or neurospheres that have been differentiated from stem cells (e.g., mammalian stem cells) with a candidate drug, wherein a first set of neuronal cells or spheres have been differentiated from stem cells (i.e., induced pluripotent stem cells, embryonic stem cells) that have mutation(s) affecting the normal expression of a gene from the chromosome and optionally, a second set of neurons that are differentiated from stem cells that do not have said mutation(s); and/or (b) contacting set(s) of neurospheres that have been derived from stem cells (e.g., mammalian stem cells) with the candidate drug, wherein a first set of neurospheres comprises neurospheres that have been differentiated from the stem cells that have mutation(s) affecting the normal expression of the gene from an X-chromosome, and optionally, a second set of neurospheres that have been differentiated from stem cells that do not have said mutation(s); and/or (c) contacting set(s) of cortical organoids that have been generated from stem cells with the candidate drug, wherein a first set of cortical organoids have been differentiated from stem cells that have mutation(s) affecting the normal expression of the gene from an X-chromosome, and optionally, a second set of cortical organoids that have been differentiated from stem cells that do not have said mutation(s); (d) evaluating whether the candidate drug rescues or ameliorates deleterious biological effect(s) resulting from abnormal expression of a gene from an X-chromosome in the first set of neurons, and/or the first set of neurospheres, and/or the first set of cortical organoids. In yet another embodiment, the neural drug screening platform, further comprises determining whether the candidate drug is toxic or negatively effects biological activity in neurons, neurospheres, and/or cortical organoids that do not have mutation(s) in the gene being studied. The mutations in the gene can be nonsense mutations, frameshift mutations, loss of function point mutations, etc. In a particular embodiment, the gene to be screened is CDKL5, which is associated with CDD. As the screening methods are directed to neural cells, the biological activity to be evaluated by the candidate drug can induce calcium oscillations, synaptic activity, and/or synaptic morphology in neurons, neural-like cells or organoids. The level of mosaicism can be controlled based upon the ratio of “normal” stem cells to “mutated” stem cells being differentiated into the neurospheres or other neural cell aggregation.

The disclosure further provides for a pharmaceutical composition comprising therapeutic compound(s) comprising Ivabradine, Solifenacin, AZD1080, Crenigacestat, salts of the foregoing and combinations thereof disclosed herein. Any of a variety of art-known methods can be used to administer a therapeutic compound(s) comprising Ivabradine, Solifenacin, AZD1080, Crenigacestat and combinations thereof disclosed herein. For example, administration can be parenterally, by injection or by gradual infusion over time. The therapeutic compound(s) alone or with the other therapeutic agents can be administered orally, intravenously, intraperitoneally, intramuscularly, subcutaneously, intracavity, intracranially, intraspinally, by inhalation, or transdermally.

A pharmaceutical composition comprising therapeutic compound(s) of the disclosure can be in a form suitable for administration to a subject using carriers, excipients, diluents and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The disclosure further provides for a pharmaceutical composition comprising Ivabradine, Solifenacin, AZD1080, Crenigacestat and combinations thereof therapeutic compound(s) disclosed herein that can be administered in a convenient manner, such as by injection (subcutaneous, intravenous, intracranial, intraspinal etc.), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

A “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the individual's diet. For oral therapeutic administration, the pharmaceutical composition can be incorporated with excipients and used in the form of ingestible tablets, buccal tablets, troches, capsules, elixirs, suspensions, syrups, wafers, chewable tablets, gummies, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic/biocompatible in the amounts employed. In addition, the pharmaceutical composition can be incorporated into sustained-release preparations and formulations.

In further embodiments, the disclosure further provides that oral pharmaceutical formulations comprising therapeutic compound(s) disclosed herein may have an enteric coating. As used herein “enteric coating”, is a material, a polymer material or materials which encase the medicament core (e.g., prodrug the disclosure). Typically, a substantial amount or all of the enteric coating material is dissolved before the medicament or therapeutically active agent is released from the dosage form, so as to achieve delayed dissolution of the medicament core. A suitable pH-sensitive polymer is one which will dissolve in intestinal juices at a higher pH level (pH greater than 6), such as within the small intestine and therefore permit release of the pharmacologically active substance in the regions of the small intestine and not in the upper portion of the GI tract, such as the stomach. An “enterically coated” drug or tablet refers to a drug or tablet that is coated with a substance—i.e., with an “enteric coating”—that remains intact in the stomach but dissolves and releases the drug once the small intestine is reached.

The coating material is selected such that the therapeutically active agent will be released when the dosage form reaches the small intestine or a region in which the pH is greater than pH 6. The coating may be a pH-sensitive material, which remains intact in the lower pH environs of the stomach, but which disintegrate or dissolve at a more neutral pH commonly found in the small intestine of the patient. For example, the enteric coating material begins to dissolve in an aqueous solution at pH between about 6 to about 7.4.

Enteric coatings include, but are not limited to, beeswax and glyceryl monostearate; beeswax, shellac and cellulose; and cetyl alcohol, mastic and shellac, as well as shellac and stearic acid (U.S. Pat. No. 2,809,918); polyvinyl acetate and ethyl cellulose (U.S. Pat. No. 3,835,221); and neutral copolymer of polymethacrylic acid esters (Eudragit L30D) (F. W. Goodhart et al., Pharm. Tech., pp. 64-71, April 1984); copolymers of methacrylic acid and methacrylic acid methylester (Eudragits), or a neutral copolymer of polymethacrylic acid esters containing metallic stearates (Mehta et al., U.S. Pat. Nos. 4,728,512 and 4,794,001). Such coatings comprise mixtures of fats and fatty acids, shellac and shellac derivatives and the cellulose acid phthalates, e.g., those having a free carboxyl content. See, Remington's at page 1590, and Zeitova et al. (U.S. Pat. No. 4,432,966), for descriptions of suitable enteric coating compositions.

In another embodiment, the therapeutic composition can be formulated as an immediate release formulation such that it is delivered in the upper gastrointestinal tract. In still another formulation the therapeutic composition can comprise a gastroretentive formulation comprising, e.g., carbopol or some other hydrophilic polymer.

Preparations for parenteral administration of a composition comprising therapeutic compound(s) of the disclosure include sterile aqueous or non-aqueous solutions, suspensions, and emulsions. Examples of non-aqueous solvents are propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives such as, other antimicrobial, anti-oxidants, cheating agents, inert gases and the like also can be included.

Pharmaceutical compositions suitable for injectable use include sterile aqueous solutions (where water soluble) or dispersions and sterile powders for the extemporaneous preparation of sterile injectable solutions or dispersions. In all cases, the composition should be sterile and should be fluid to the extent that easy syringability exists. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be typical to include isotonic agents, for example, sugars, polyalcohols, such as mannitol, sorbitol, or sodium chloride in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

It is especially advantageous to formulate parenteral compositions in dosage unit form for ease of administration and uniformity of dosage. “Dosage unit form” as used herein, refers to physically discrete units suited as unitary dosages for the individual to be treated; each unit containing a predetermined quantity of pharmaceutical composition is calculated to produce the desired therapeutic effect in association with the required pharmaceutical carrier. The specification for the dosage unit forms of the disclosure are related to the characteristics of the pharmaceutical composition and the particular therapeutic effect to be achieve.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

The pharmaceutical compositions according to the disclosure may be administered at a therapeutically effective amount either locally or systemically. As used herein, “administering a therapeutically effective amount” is intended to include methods of giving or applying a pharmaceutical composition of the disclosure to a subject that allow the composition to perform its intended therapeutic function. The therapeutically effective amounts will vary according to factors, such as the degree of infection in a subject, the age, sex, and weight of the individual. Dosage regime can be adjusted to provide the optimum therapeutic response. For example, several divided doses can be administered daily or the dose can be proportionally reduced as indicated by the exigencies of the therapeutic situation.

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more therapeutic compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise therapeutic compounds disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The following examples are intended to illustrate but not limit the disclosure. While they are typical of those that might be used, other procedures known to those skilled in the art may alternatively be used.

### Examples

Participant recruitment. Skin biopsies from three female and three male subjects diagnosed with CDD, and their first-degree related healthy control (mother or father, respectively) were obtained.

Call reprogramming. Fibroblasts from CDD patients and their respective non-affected controls were reprogrammed using non-integrative methods, either by transduction with six episomal plasmid vectors (Sox2, Klf4, Oct3/4, Lin28, p53 shRNA and L-Myc) or by Sendai virus vector-mediated expression of Oct4, Sox2, Klf-4 and c-myc (CytoTune-iPS Kit, Life Technologies). iPSCs from each subject were isolated and transferred to feeder-free Matrigel® (BD Biosciences) coated plates approximately four weeks after initial transduction. Colonies were propagated in mTeSR1 (Stem Cell Technologies) and manually passaged as small colonies. Standard G-banding karyotype of iPSC clones was performed. Analysis of copy number variation in iPSC clones was performed.

Generation of neural progenitor cells (NPCs) and 2D neurons. iPSCs were differentiated into NPCs as previously described (Thomas et al., Cell Stem Cell, 21:319-331, e318, 2017; Chailangkarn et al., Nature, 2016). Briefly, iPSCs colonies were cultured for 2 days in the presence of DMEM/F12 1:1 with 1×Glutamax (Life Technologies), 1×N2 NeuroPlex (N2; Gemini Bio-products), lx penicillin-streptomycin (PS; Life Technologies), 10 μm SB431542 (SB; StemGent) and 1 μm dorsomorphin (Dorso; Tocris Biosciences). The colonies were lifted off and kept in suspension, under rotation (95 rpm) for 7 days to form embryoid bodies (EB). EBs were gently disrupted, plated onto Matrigel-coated plates and cultured in DMEM/F12 1:1 with 1×HEPES, 1×Glutamax, 1×PS, 0.5×N2, 0.5×Gem21 NeuroPlex (Gem21; Gemini Bio-products), supplemented with 20 ng/mL basic fibroblast growth factor (bFGF; Life Technologies) for 7 days. Next, neural rosettes were manually collected, dissociated with Accutase (Thermo Fisher), and NPCs were plated onto poly-L-ornithine/laminin-coated plates. NPCs were expanded in the presence of bFGF and fed every other day. Neural differentiation was promoted by bFGF withdrawn from the medium; ROCK inhibitor (Y-27632; Calbiochem, Sigma-Aldrich) was added at 5 μm for the first 2 days. Medium was changed twice a week and cells were allowed to differentiate for as long as needed.

Generation of cortical organoids. Cortical organoids were generated as previously described (Trujillo et al., BioRxiv, 358622, 2018). Briefly, fully-grown iPSCs colonies were dissociated for approximately 10 min at 37° C. with Accutase in PBS (1:1), and centrifuged for 3 min at 150×g. The cell pellet was resuspended in mTeSR1 supplemented with 10 μM SB and 1 μM Dorso. Approximately 4×10⁶ cells were transferred to one well of a 6-well plate and kept in suspension under rotation (95 rpm) in the presence of 5 μM ROCK inhibitor for 24 h to form free-floating spheres. After 3 days, mTeSR1 was substituted by Medial [Neurobasal (Life Technologies) supplemented with 1×Glutamax, 1×Gem21, 1×N2, 1×MEM nonessential amino acids (NEAA; Life Technologies), 1×PS, 10 μM SB and 1 μM Dorso] for 7 days. Cells were then maintained in Media2 [Neurobasal with 1×Glutamax, 1×Gem21, 1×NEAA and 1×PS] supplemented with 20 ng/mL bFGF for 7 days, followed by 7 additional days in Media2 supplemented with 20 ng/mL of FGF2 and 20 ng/mL EGF (PeproTech). Next, cells were transferred to Media3 [Media2 supplemented with 10 μg/mL of BDNF, 10 μg/mL of GDNF, 10 μg/mL of NT-3 (all from PeproTech), 200 μM L-ascorbic acid and 1 mM dibutyryl-cAMP (Sigma-Aldrich)]. After 7 days, cortical organoids were maintained in Media2 for as long as needed, with media changes every 3-4 days.

Mycoplasma testing. All cellular cultures were routinely tested for mycoplasma by PCR. Media supernatants (with no antibiotics) were collected, centrifuged, and resuspended in saline buffer. Ten microliters of each sample were used for a PCR. Only negative samples were used in the study.

Proteomics and phosphoproteomics analysis. Cell lysates from NPCs, neurons, and cortical organoids were prepared with RIPA buffer or 100 mM TEAB with 1% SDS. After reduction (10 mM TCEP) and alkylation (50 mM chloroacetamide), MeOH/CHCl₃ precipitation was performed. Pellets were dissolved with 6 M urea in 50 mM TEAB, and LysC/Tryp (Promega) was added at 1:25 (w/w) ratio. After 3-4 h incubation at 37° C., reaction mixture was diluted with 50 mM TEAB for urea to be less than 1 M. After overnight digestion, peptides were labelled with TMT 10-plex (Thermo Fisher), followed by quenching with hydroxylamine. All reaction mixtures were pooled together and dried using SpeedVac. One hundred μg of peptides were separated for total protein analyses, and the remaining mixtures were used for phosphoproteomic analyses. After desalting using Pierce peptide desalting spin columns (Thermo Fisher), phosphopeptides were enriched using High-Select TiO₂ enrichment kit (Thermo Fisher). Resulting eluates were dried in SpeedVac immediately after the enrichment. Peptides to be analyzed in both total and phosphoprotein analyses were fractionated using Pierce High pH reversed-phase peptide fractionation kit (Thermo Fisher) and then dried in SpeedVac. Dried peptides were dissolved with buffer A (5% acetonitrile, 0.1% formic acid), and each fraction was injected directly onto a 25 cm, 100 μm-ID columns packed with BEH 1.7 μm C18 resin (Waters). Samples were separated at a flow rate of 300 nL/min on nLC 1000 (Thermo Fisher). A gradient of 1-25% buffer B (80% acetonitrile, 0.1% formic acid) over 200 min, an increase to 50% B over 120 min, an increase to 90% B over another 30 min and held at 90% B for a final 10 min of washing was used for 360 min total run time. Column was re-equilibrated with 20 μL of buffer A prior to the injection of sample. Peptides were eluted directly from the tip of the column and nanosprayed directly into the mass spectrometer Orbitrap Fusion by application of 2.8 kV voltage at the back of the column. Fusion was operated in a data dependent mode. Full MS1 scans were collected in the Orbitrap at 120 k resolution. The cycle time was set to 3 s, and within this 3 s the most abundant ions per scan were selected for CID MS/MS in the ion trap. MS3 analysis with multi-notch isolation (SPS3) was utilized for detection of TMT reporter ions at 60 k resolution. Monoisotopic precursor selection was enabled, and dynamic exclusion was used with exclusion duration of 10 s. Tandem mass spectra were extracted from the raw files using RawConverter with monoisotopic peak selection.

The spectral files from all fractions were uploaded into one experiment on Integrated Proteomics Applications (IP2, Ver.5.1.3) pipeline. Proteins and peptides were searched using ProLuCID and DTASelect 2.0 on IP2 against the UniProt H. sapiens protein database with reversed decoy sequences (UniProt_Human_reviewed_05-05-2016_reversed.fasta). Precursor mass tolerance was set to 50.0 ppm, and the search space allowed all fully-tryptic and half-tryptic peptide candidates without limit to internal missed cleavage and with a fixed modification of 57.02146 on cysteine and 229.1629 on N-terminus and lysine. Peptide candidates were filtered using DTASelect parameters of −p 1 (proteins with at least one peptide are identified) −y 1 (partial tryptic end is allowed) −pfp 0.01 (protein FDR<1%) −DM 5 (highest mass error 5 ppm) −U (unique peptide only). Quantification was performed by Census on IP2. The expression value for each protein was calculated by adding the peptide-level reporter ion intensities normalized to total intensity of each channel to remove the variances caused by the different loading amount or labeling efficiency for different channels. For phosphoproteome analysis, search parameters included differential modification of 79.966331 on serine, threonine, and tyrosine, and DTASelect parameters −p 1 −y 1 −pfp 0.01 −DM 5 −m 0 (only phosphorylated peptides). To detect the phosphorylation-specific changes, peptide-level reporter ion intensities were normalized to total protein intensities.

Amino acid starvation. NPCs were grown to 80-90% confluency in complete culture medium before amino acid withdrawal. Next, medium was replaced by a glucose-containing starvation buffer, Earle's Balanced Salt Solution (EBSS) (Thermo Fisher), and the cells were incubated for 10, 30, 60, 120 or 240 minutes before protein extraction.

Western blotting. Protein was extracted using RIPA Lysis and Extraction buffer (Thermo Fisher) containing cOmplete ULTRA mini protease inhibitor (Roche) and PhosSTOP phosphatase inhibitor (Roche). Twenty μg of protein lysates were separated on a 4%-12% Bis-Tris protein gel (Novex), and transferred onto a nitrocellulose membrane (Novex) using the iBlot2 Gel Transfer device (Thermo Fisher). Following blockage with Rockland Blocking Buffer (Rockland), the membrane was incubated with primary antibodies overnight at 4° C. Next, the membrane was washed five times (5 min each) with 0.1% Tween 20 in PBS and incubated with secondary antibodies for 2 h at room temperature. Antibodies used in this study can be found in Table 1. Odyssey CLx imaging system (Li-Cor) was used for signal detection and semi-quantitative analysis was performed using Odyssey Image Studio software.

TABLE 1
Antibodies used

| | | Catalog |
|---|---|---|
| Antibody | Source | Number |
| Rabbit anti-Lin28 | Abcam | ab46020 |
| Goat anti-Nanog | R&D Systems | aF1997 |
| Mouse anti-Nestin | Abcam | ab22035 |
| Rabbit anti-Sox2 | Cell Signalling | 2748 |
| Rabbit anti-GFAP | DAKO | Z033429 |
| Chicken anti-MAP2 | Abcam | ab5392 |
| Rat anti-CTIP2 | Abcam | ab18465 |
| Rabbit anti-S6 Ribosomal | Cell Signaling | 2217 |
| Protein (5G10) | | |
| Rabbit anti-Phospho-S6 | Cell Signaling | 2215 |
| Ribosomal Protein (Ser240/244) | | |
| Rabbit anti-p70S6 Kinase | Cell Signaling | 9202 |
| Protein | | |
| Rabbit anti-Phospho-p70S6 | Cell Signaling | 9204 |
| Kinase Protein (Thr421/Ser424) | | |
| Rabbit anti-4E-BP1 Protein | Cell Signaling | 9452 |
| Rabbit anti-Phospho-4E-BP1 | Cell Signaling | 9459 |
| Protein (Thr37/46) | | |
| Mouse anti-actin | Abcam | ab8226 |
| Rabbit anti-Trimethyl- | Millipore | 07-449 |
| Histone H3 (Lys27) | | |
| Mouse anti-CDKL5 | Santa Cruz | sc-376314 |
| Chicken anti-GFP | Abcam | ab13970 |
| Rabbit anti-Synapsin1 | Millipore | AB1543P |
| Mouse anti-PSD-95 | NeuroMab | 75-028 |
| Mouse anti-Vglut1 | Synaptic Systems | 135311 |
| Rabbit anti-Homer1 | Synaptic Systems | 160003 |
| Donkey anti-Goat IgG- | Thermo Fisher | A11055 |
| Alexa Fluor 488 | | |
| Donkey anti-Mouse IgG- | Thermo Fisher | R37114 |
| Alexa Fluor 488 | | |
| Donkey anti-Rabbit IgG- | Thermo Fisher | R37118 |
| Alexa Fluor 488 | | |
| Donkey anti-Rat IgG- | Thermo Fisher | A21208 |
| Alexa Fluor 488 | | |
| Goat anti-Mouse IgM- | Thermo Fisher | A21042 |
| Alexa Fluor 488 | | |
| Goat anti-Chicken IgY- | Thermo Fisher | A11039 |
| Alexa Fluor 488 | | |
| Donkey anti-Rabbit IgG- | Thermo Fisher | A31572 |
| Alexa Fluor 555 | | |
| Donkey anti-Mouse IgG- | Thermo Fisher | A31570 |
| Alexa Fluor 555 | | |
| Donkey anti-Mouse IgG- | Thermo Fisher | A31571 |
| Alexa Fluor 647 | | |
| Donkey anti-Rabbit IgG- | Thermo Fisher | A31573 |
| Alexa Fluor 647 | | |
| Goat anti-Mouse IgM- | Thermo Fisher | A21238 |
| Alexa Fluor 647 | | |
| Goat anti-Chicken IgY- | Thermo Fisher | A21449 |
| Alexa Fluor 647 | | |

Immunofluorescence staining. iPSCs, NPCs and 2D neurons were fixed with 4% paraformaldehyde (PFA) for 15 min. After three washes with PBS, cells were permeabilized with 0.25% Triton X-100 for 15 min, blocked with 3% bovine serum albumin (BSA) and incubated overnight at 4° C. with primary antibodies diluted in 3% BSA. The following day, cells were washed and incubated with the secondary antibodies for 1 h. Antibodies used in this study can be found in Table 1. For nuclei staining, DAPI solution (1 μg/mL) was used. The slides were mounted using ProLong Gold antifade reagent and analyzed under a fluorescence microscope (Z1 Axio Observer Apotome, Zeiss).

Synaptic puncta quantification. After 8 weeks of differentiation, 2D neurons were fixed and stained. The number of pre-synaptic VGLUT1+ and post-synaptic HOMER1+ puncta co-localization was blindly quantified. Only puncta overlapping MAP2+ processes were scored. Images were taken randomly from two independent experiments.

DNA fragmentation analysis. NPCs were harvested to single cell suspension in PBS, fixed by addition of 70% ethanol and stored for 24 h at 4° C. Next, cells were washed with PBS, resuspended in DAPI staining solution (0.1% (v/v) Triton X-100, 1 μg/mL DAPI in PBS) and incubated for 5 min at 37° C. Samples were analyzed on the NC-3000 Advanced Image Cytometer (Chemometec), using the preoptimized DNA Fragmentation Assay. The amount of High Molecular Weight (HMW) DNA retained in the cells was quantified in order to detect apoptotic cells with fragmented DNA (subG1 population).

Caspase assay. Caspase activity was assessed using Green FLICA Caspases 3 & 7 Assay kit (ImmunoChemistry Technologies, LLC) according to manufacturer's protocol. Briefly, NPCs were harvested to single cell suspension, washed with PBS and stained with 1× carboxyfluorescein Fluorochrome Inhibitor of Caspase Assay (FAM-FLICA) reagent, 10 μg/mL Hoechst 33342 and 10 μg/mL propidium iodide (PI). Samples were analyzed on the NC-3000 Advanced Image Cytometer (Chemometec) using the pre-optimized Caspase Assay. PI stained the nonviable cell population whereas FAM-FLICA stained cells with caspase activity for apoptosis analysis.

Proliferation assay. NPC proliferation was assessed by cell counting. Briefly, a pre-determined number of NPCs was plated onto poly-L-ornithine/laminin-coated plates (day 0). After 4 h, the plates were transferred to a Viva View FL Incubator Microscope (Olympus), and allowed to acclimatize for 30 min (TO). Next, the cellular proliferation was monitored and contrast-phase images were taken after 48 h (T48; day2). The images were processed and the number of cells at TO and T48 was determined using the Cell Counter plugin on the Fiji platform. The difference between day 2 and day 0 was used to estimate the NPC proliferation rate.

Mitochondrial depolarization. Disruption of the mitochondrial transmembrane potential (Dym) is usually associated with early stages of apoptosis. We measured Dym in NPCs using the cationic dye JC-1. Briefly, NPCs were harvested to single cell suspension in PBS containing 2.5 μg/mL of a JC-1 solution, and cells were incubated for 10 min at 37° C. After washes with PBS, DAPI (1 μg/mL) was added for nuclei staining. Samples were analyzed on the NC-3000 Advanced Image Cytometer (Chemometec) using the preoptimized Mitochondrial Potential Assay. At high concentrations JC-1 forms aggregates and become red fluorescent, while in apoptotic cells the mitochondrial potential collapses and JC-1 localizes to the cytosol in its monomeric green fluorescent form. The number of cells with collapsed mitochondrial membrane potential was quantified and the mitochondrial depolarization estimated as a decrease in the red/green fluorescence intensity ratio.

Neuronal spine-like dynamics. Dendrites from 8-week-old neurons stably transfected to express enhanced GFP by Synapsin1 promoter self-inactivating lentivirus were recorded using a Z1 Axio Observer Apotome (Zeiss). Images were taken every 30 sec for 1 h, and analyzed using the NeuronJ plugin on the Fiji platform. Only neurons that displayed at least two visible neurites at t=0 and had changes in spine dynamics during the 60 min time course were analyzed.

Cellular migration. Three-week old spheres were treated for 3 additional weeks with 1 μM of selected compounds. Cellular migration was evaluated 8 days after spheres were plated onto poly-L-ornithine/laminin-coated plates. The outward radial migration was measured using NeuronJ plugin on the Fiji platform.

Quantitative Multiplex Co-Immunoprecipitation (QMI). QMI analysis was performed. Briefly, cortical organoids were homogenized in lysis buffer [150 mM NaCl, 50 mM Tris (pH 7.4), 1% NP-40, 10 mM NaF, 2 mM sodium orthovanadate+Protease/phosphatase inhibitor cocktails (Sigma)] using a glass tissue homogenizer, incubated for 15 min, centrifuged at high speeds to remove nuclei and debris, and protein concentration was determined using a Pierce BCA kit (Thermo Fisher). A master mix containing equal numbers of each antibody-coupled Luminex bead class was prepared and distributed into post-nuclear cell lysate samples in duplicate. Protein complexes were immunoprecipitated from samples containing equal amounts of protein overnight at 4° C., washed twice in ice-cold Fly-P buffer [50 mM tris (pH 7.4), 100 mM NaCl, 1% bovine serum albumin, and 0.02% sodium azide], and distributed into as many wells of a 96-well plate as there were probes, on ice. Biotinylated detection antibodies were added and incubated for 1 h, with gentle agitation at 500 rpm in a cold room (4° C.). Following incubation, microbeads and captured complexes were washed three times in Fly-P buffer using a Bio-Plex Pro II magnetic plate washer in a cold room. Microbeads were then incubated for 30 min with streptavidin-PE on ice, washed three times, and resuspended in 125 μL of ice-cold Fly-P buffer. Fluorescence data were acquired on a customized, refrigerated Bio-Plex 200 instrument according to the manufacturer's recommendations.

Data preprocessing and inclusion criteria XML output files were parsed to acquire the raw data for use in MATLAB while XLS files were used for input into R statistical packages. For each well from a data acquisition plate, data were processed to (i) eliminate doublets on the basis of the doublet discriminator intensity (>5000 and <25,000 arbitrary units; Bio-Plex 200), (ii) identify specific bead classes within the bead regions used, and (iii) pair individual bead PE fluorescence measurements with their corresponding bead regions. This processing generated a distribution of PE intensity values for each pairwise protein co-association measurement. ANC Adaptive nonparametric analysis with empirical alpha cutoff (ANC) was used to identify high-confidence, statistically significant differences (corrected for multiple comparisons) in bead distributions on an individual interaction basis.

Hits were required to be present in at least 6 of 8 replicates at an adjusted P<0.05. The α-cutoff value required per experiment to determine statistical significance was calculated to maintain an overall type I error of 0.05 (adjusted for multiple hypothesis testing with Bonferroni correction), with further empirical adjustments to account for technical errors. CNA: Bead distributions used in ANC were collapsed into a single median fluorescent intensity (MFI), which was averaged across duplicate samples and input into the WGCNA package for R. Data were filtered to remove weakly detected interactions (‘noise’, MFI<100), and batch effects were removed using the COMBAT function for R, with “experiment number” as the “batch” input. Post-Combat data was log 2 transformed prior to CNA analysis. Closely related protein co-associations were assigned to arbitrary color-named modules by the WGCNA program. Modules whose eigenvectors significantly (P<0.05) correlated with the genotype were considered significant to produce a high-confidence set of interactions that were both individually significantly different in comparisons between experimental groups, and that belonged to a larger module of co-regulated interactions that was significantly correlated with experimental group. Hierarchical Clustering was performed using pvlcust in R.

NPC transplantation. Human iPSC-derived NPCs were stably transfected to express EGFP by Synapsin1 promoter self-inactivating lentivirus. Ten to twenty thousand cells were injected per site, 1 mm from the midline between the Bregma and Lambda and 1-2 mm deep into the cortex and striatum of newborns immunosuppressed NOD/SCID mice. Briefly, newborns (P0-P2) were anesthetized by hypothermia and then placed in a contoured Styrofoam mold. Two microliters of NPCs were injected into both hemispheres using a 5 ml Hamilton syringe with a 32-gauge needle. After 6 months, injected animals were anesthetized and perfused. Entire brains were sliced using a cryomicrotome and immunohistochemical analysis were carried out on free-floating mice brain slices to identify and evaluate the efficiency of the transplantation.

Postmortem brain specimens and cortical sampling. Four postmortem brains that were gender, age and hemisphere matched were used. Specifically, postmortem brain tissue from a 5-year-old female CDD patient with a Pro719CysfsX66 mutation and, a 30-year-old female CDD patient with a deletion comprehending exons 1-3 in the CDKL5 gene; and two female individuals with no described genetic alteration (control), respectively at 6- and 30-year-old. All brain specimens were harvested within a postmortem interval of 15-36 h and had been immersed and fixed in 10% formalin for less than 3 years. For the purpose of the present experiments, samples were obtained from anatomically well-identified cortical areas in a consistent manner across specimens, comprehending the primary somatosensory cortex (Brodmann area 3), the primary motor cortex (Brodmann area 4) and the secondary visual area (Brodmann area 18). Details about the tissue processing protocol is provided online by the NIH NeuroBioBank. These parts of the cortex were focused on because pathologies in dendritic morphology in these areas have been reported in other neurodevelopmental disorders. In addition, pyramidal neurons in the selected areas reach their mature-like morphology early in development and start displaying dendritic pathologies sooner than high integration areas, such as the prefrontal cortex, allowing for a comparison of post-mortem findings with iPSC-derived neurons in early stages of development. Samples were obtained from Harvard Brain Tissue Resource Center, the University of Maryland Brain and Tissue Bank, and the University of Miami Brain Endowment Bank, which are Brain and Tissue Repositories of the NIH NeuroBioBank.

Postmortem brain tissue processing. Processing and staining of brain tissue samples was performed using the semi-rapid Golgi technique. Briefly, specimens were immersed in a solution of 1% silver nitrate for ten days. Blocks were then sectioned on a vibratome, perpendicular to the pial surface, at a thickness of 110-120 μm. Golgi sections were cut into 100% ethyl alcohol and transferred briefly into toluene, mounted onto glass slides and cover-slipped.

Golgi-impregnated neurons. Neurons included in the morphological analysis did not display degenerative changes. The morphological analysis was performed on pyramidal neurons located in cortical layers V/VI, with fully impregnated soma, apical dendrites with present oblique branches, and at least two basal dendrites with second/third order segments. To minimize the effects of cutting on dendritic measurements, we included neurons with cell bodies located near the center of 120-μm thick histological sections, with natural terminations of higher-order dendritic branches present where possible. Inclusion of the neurons completely contained within 120-μm sections biases the sample toward smaller neurons, leading to the underestimation of dendritic length; therefore, the same criteria blinded across all control and CDD specimens was used, and thus included the neurons with incomplete endings if they were judged to otherwise fulfill the criteria for successful Golgi impregnation. All neurons were oriented with apical dendrite perpendicular to the pial surface; inverted pyramidal cells as well as magno-pyramidal neurons were excluded from the analysis.

Postmortem brain neuronal tracing. Neuronal morphology was quantified along x-, y-, and z-coordinates using OPTIMAS Bioscan software (Media Cybernetics Inc., MD, USA) connected to a Zeiss Axio scope system, equipped with 100×/1.25 oil Plan Fluor objective and a CCD camera (Hamamatsu, ORCA-Flash4.0 V3), motorized X, Y, and Z-focus for high-resolution image acquisition and digital quantitation. Tracings were conducted on both apical and basal dendrites, and the results reflect summed values for both types of dendrites per neuron. Following the recommendation that the applications of Sholl's concentric spheres for the analysis of neuronal morphology are not adequate when neuronal morphology is analyzed in three dimensions, dendritic tree analysis was conducted with the following measurements: (1) soma area—cross sectional surface area of the cell body; (2) dendritic length—summed total length of all dendrites per neuron; (3) dendrite number—number of dendritic trees emerging directly from the soma per neuron; (4) dendritic segment number—total number of segments per neuron; (5) dendritic spine/protrusion number—total number of dendritic spines per neuron; (6) dendritic spine/protrusion density—average number of spines/20 μm of dendritic length; and (7) branching point number—number of nodes (points at the dendrite where a dendrite branches into two or more) per neuron. Dendritic segments were defined as parts of the dendrites between two branching points—between the soma and the first branching point in the case of first order dendritic segments, and between the last branching point and the termination of the dendrite in the case of terminal dendritic segments. Since the long formalin-fixation time may result in degradation of dendritic spines, spine values may be underestimated and are thus reported here with caution. All of the tracings were accomplished blind to brain region and diagnostic status.

iPSC-derived neuronal tracing. The iPSC-derived samples consisted of EGFP-SYN1-positive 8-week-old neurons with pyramidal- or ovoid-shaped soma and at least two branched neurites (dendrites) with visible spines/protrusions. Protrusions from dendritic shaft, which morphologically resembled dendritic spines in postmortem specimens, were considered and quantified as dendritic spines in iPSC-derived neurons. The neurites were considered dendrites based on the criteria applied in postmortem studies: (1) thickness that decreased with the distance from the cell body; (2) branches emerging under acute angle; and (3) presence of dendritic spines. Only EGFP-positive neurons with dendrites displaying evenly distributed fluorescent stain along their entire length were considered. In addition, neurons included in the analysis had to exhibit the nuclei co-stained with CTIP2, indicative of layer V/VI neurons. The morphology of the neurons was quantified along x-, y-, and z-coordinates using Neurolucida v.9 software (MBF Bioscience, Williston, VT) connected to a Nikon Eclipse E600 microscope with 40× oil objective. No distinction was made between apical and basal dendrites, and the results reflect summed length values of all neurites/dendrites per neuron, consistent with what was done for the postmortem neurons. The same set of measurements used in the analysis of Golgi-impregnated neurons was applied to the analysis of iPSC-derived neurons, and all tracings were accomplished blind to the genotype.

Multi-electrode array (MEA) recording. Six-week-old cortical organoids were plated per well in poly-L-ornithine/laminin-coated 12-well MEA plates (Axion Biosystems). Cells were fed twice a week and measurements were collected 24 h after the medium was changed, once a week, starting at two weeks after plating (8 weeks of organoid differentiation). Recordings were performed using a Maestro MEA system and AxIS Software Spontaneous Neural Configuration (Axion Biosystems) with a customized script for band-pass filter (0.1-Hz and 5-kHz cutoff frequencies). Spikes were detected with AxIS software using an adaptive threshold crossing set to 5.5 times the standard deviation of the estimated noise for each electrode (channel). The plate was first allowed to rest for 3 min in the Maestro device, and then 3 min of data were recorded. For the MEA analysis, the electrodes that detected at least 5 spikes/min were classified as active electrodes using Axion Biosystems' Neural Metrics Tool. Bursts were identified in the data recorded from each individual electrode using an inter-spike interval (ISI) threshold requiring a minimum number of 5 spikes with a maximum ISI of 100 ms. At least 10 spikes under the same ISI with a minimum of 25% active electrodes were required for network bursts in the well. The synchrony index was calculated using a cross-correlogram synchrony window of 20 ms. Independent experiments were performed with three cell lines with at least 3 technical replicates.

Cellular electrophysiology. Whole-cell patch-clamp recordings were performed in cultured human iPSC-derived neurons at room temperature (˜20° C.). The extracellular solution for patch-clamp experiments contained the following: 130 mM NaCl, 3 mM KCl, 1 mM CaCl₂, 1 mM MgCl₂, 10 mM HEPES, and 10 mM glucose; pH 7.4 with 1 M NaOH (˜4 mM Na+ added). The internal solution for patch electrodes contained the following: 138 mM K-gluconate, 4 mM KCl, 10 mM Na₂-phosphocreatine, 0.2 mM CaCl₂, 10 mM HEPES (Na⁺ salt), 1 mM EGTA, 4 mM Mg-ATP, 0.3 mM Na-GTP; pH 7.4 with 1 M KOH (˜3 mM K⁺ added). The osmolarity of all solutions was adjusted to 290 mOsm. Electrodes for electrophysiological recording were pulled on a Flaming/Brown micropipette puller (Model P-87, Sutter Instrument) from filamented borosilicate capillary glass (1.2 mm OD, 0.69 mm ID, World Precision Instruments). The electrode resistances were 3-6 MΩ for whole-cell recordings. Patch clamp experiments were performed with an Axon CV-4 headstage, Axopatch 200A amplifier and DigiData 1322A (Molecular Devices). Recordings were digitized at 10 kHz and low-pass filtered at 2 kHz. The spontaneous excitatory synaptic currents were recorded with the holding membrane potentials of −60 mV and last 3-5 mins for each neuron. Evoked action potentials were measured from −60 mV under current-clamp configuration. Data were all corrected for liquid junction potentials (10 mV). Voltage-clamp synaptic currents (sEPSCs) were analyzed using Mini Analysis (Synaptosoft) and other electrophysiological results were analyzed using pCLAMP 10 software (Molecular Devices). The electrophysiological experiments and analyses were performed in a blinded manner to avoid bias.

CDD High Throughput Screening (HTS) platform development. An HTS platform for CDD was developed. Briefly, the HTS system relies on spheroids derived from human iPSCs that comprise a balanced culture of cortical neurons and astrocytes. These spheroids display quantifiable, robust and uniform spontaneous calcium oscillations which correlated with synchronous neuronal activity. The CDD HTS platform uses cells derived from CDD1 and Controll lines.

Chemical library and drug treatment. The compound library used in this study comprises a unique collection of 1112 compounds with biological activity used for neurologic research and associated assays; of which two-thirds are FDA-approved drugs (SelleckChem, Houston, TX, USA). Three-week-old CDD spheroids received chronic treatment three-times per week, during three weeks. All compounds were tested at 1 μM in three technical replicates; vehicle controls (DMSO or water) were included in multiple replicates. The dose-response assay comprehended six concentrations ranging from 0.0003 to 1 μM, in four technical replicates.

HT Calcium oscillation assay. To assess the intracellular calcium oscillations in CDD and control cortical spheroids, cells were incubated with the FLIPR Calcium 6 Kit (Molecular Devices LLC, San Jose, CA, USA). Briefly, cells were pre-loaded with Calcium 6 Dye for 2 h prior to the recording. The dye interacts with intracellular calcium and changes in fluorescence occur according to increased or decreased calcium concentration. The peaks observed correlate with synchronous neural activity in the spheroids. The kinetics of intracellular calcium oscillations was determined using the FLIPR Tetra High-Throughput Cellular Screening System (Molecular Devices LLC): the fluorescence intensity was set at 515-575 nm following excitation at 470-495 nm for 10 min at a frequency of 3 Hz; the exposure time per read was 0.4 s, the gain was set to 30, and the excitation intensity was set to 40%. The instrument temperature was kept at 37° C.

HT cell viability. To determine any cytotoxic effect of the drug treatment on the neural culture, the CellTiter Glo 3D Cell Viability Assay (Promega, Madison, WI, USA) was performed according to the manufacturer's instructions. Briefly, following the intracellular calcium oscillation recording, the spheroids were extensively washed with PBS for removal of the calcium dye. Next, cells were incubated with CellTiter Glo Reagent for 30 min at room temperature and the number of viable cells was estimated based on the amount of ATP present in the culture. The luminescent signal was captured using a PHERAstar FSX Microplate Reader (BMG Labtech, Ortenberg, Germany).

HT 3D imaging for size measurement. To determine the size of CDD and control spheroids, contrast-phase images from the 384-well plates were weekly acquired using the ImageXpress Microscope-Micro Confocal System (Molecular Devices LLC). The instrument temperature was kept at constant 37° C. during image acquisition.

HT screening analysis. A multiparametric analysis of representative descriptors of the intracellular calcium oscillation was performed. The features used include: 1. Peak count; 2. Average (Avg) Peak Height (Amplitude); 3. Peak Height standard deviation (s.d.); 4. Avg Peak Width, 5. Peak Width s.d.; 6. Avg Peak Spacing; 7. Peak Spacing s.d.; 8. Avg Peak Rise Time; 9. Peak Rise Time s.d.; 10. Avg Peak Decay Time; 11. Peak Decay Time s.d.; 12. Class 1 Peak Count; 13. Class 2 Peak Cound; 14. Class 3 Peak Count; 15. Singular Peak Count; 16. Irregular Peak Count, and 17. Subpeak Count. For peak classification, Class 1 Peak display 100-66% of max peak height, while Class 2 and Class 3, 66-33% and 33-15%, respectively. To determine the Scalar Perturbation, the data was normalized by dividing raw parameters by the median values of controls from the same plate. Each parameter was standardized by z-score of normalized and log-transformed control values. Next, the Euclidean norm between selected parameters was calculated and averaged the Scalar Perturbation score among replicates. In the Parameter Recovery, max/min boundaries from vehicle Control were determined from corresponding plate. For each replicate, we summed the number of parameter found to be within the max/min boundaries of the Control. Next, for each replicate we divided the sum of the recovered parameter by the total number of parameters and established the percentage of parameters recovered to the Control boundaries and, averaged Parameter Recovery percentages between compound replicates.

To determine the correlation between compounds, pathways and genes, the databases NCATS (National Center for Advancing Translational Sciences)—Inxight: Drugs portal (version 1.1, [https://]drugs.ncats.io/), CTD (Comparative Toxicogenomics Database), KEGG (Kyoto Encyclopedia of Genes and Genomes), and DGIdb (Drug Gene Interaction Database) were used. NCATS Inxight: Drugs is a comprehensive portal for drug development information, and contains information on ingredients in medicinal products, including US approved drugs, marketed drugs and investigational drugs. CTD is a literature-based and manually curated associations between chemicals, gene products, phenotypes, diseases and environmental exposures. KEGG is a biological encyclopedia for the understanding of high-level functions and utilizes of a biological system and its parts (cell, molecules, genes) and, DBIdb is a drug-gene interaction database, composed by consolidated interactions and druggable genes that were extracted from peer-reviewed manuscripts, databases and web resources.

Statistical analysis. Data are presented as mean±s.e.m., unless otherwise indicated. No statistical method was used to predetermine the sample size. The statistical analyses were performed using GraphPad Prism v6; two-tailed Mann-Whitney U test or unpaired t-test with multiple-comparison correction were used as indicated. Significance was defined as P<0.05(*), P<0.01(**), P<0.001(***) or P<0.0001(****). Blinding was used for comparing affected and control samples.

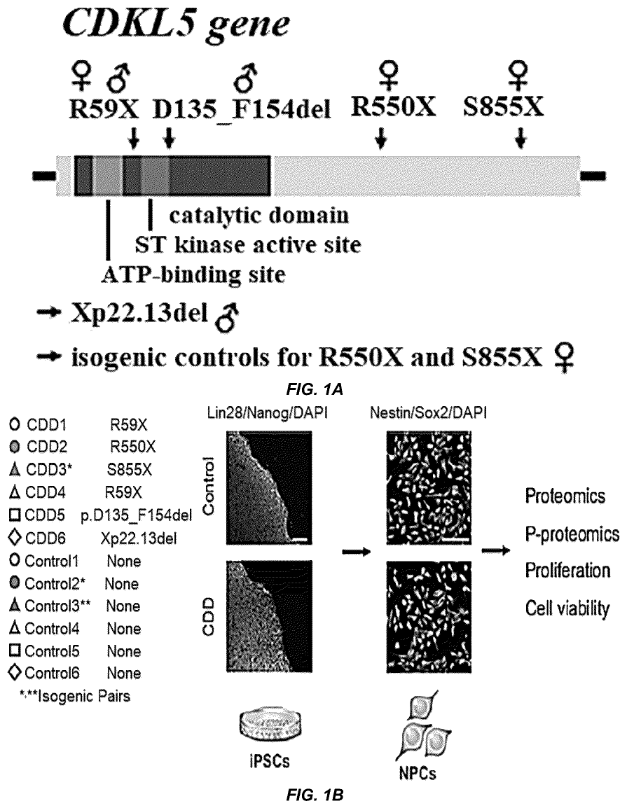

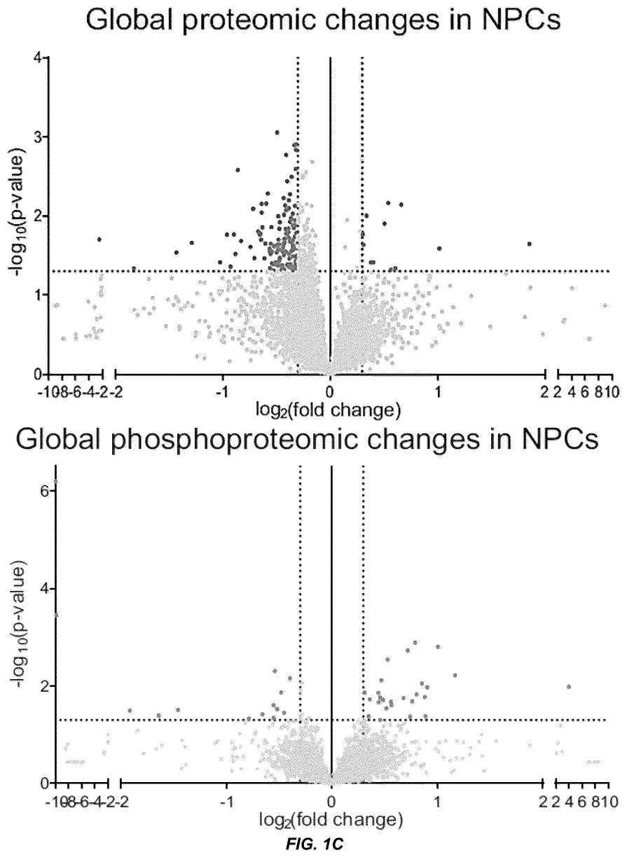

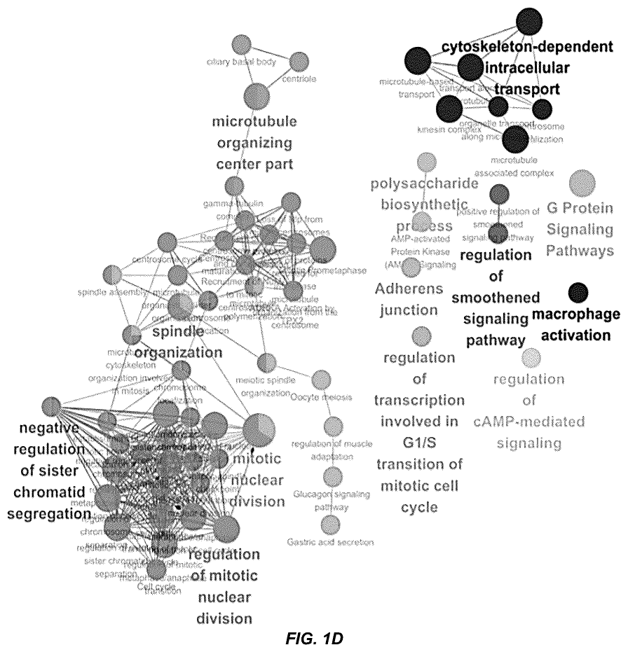

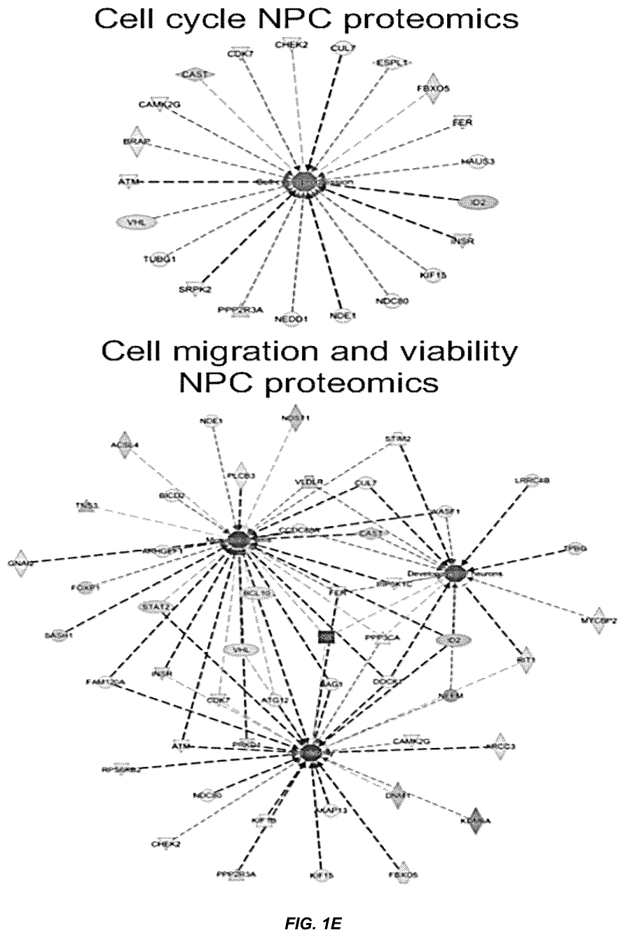

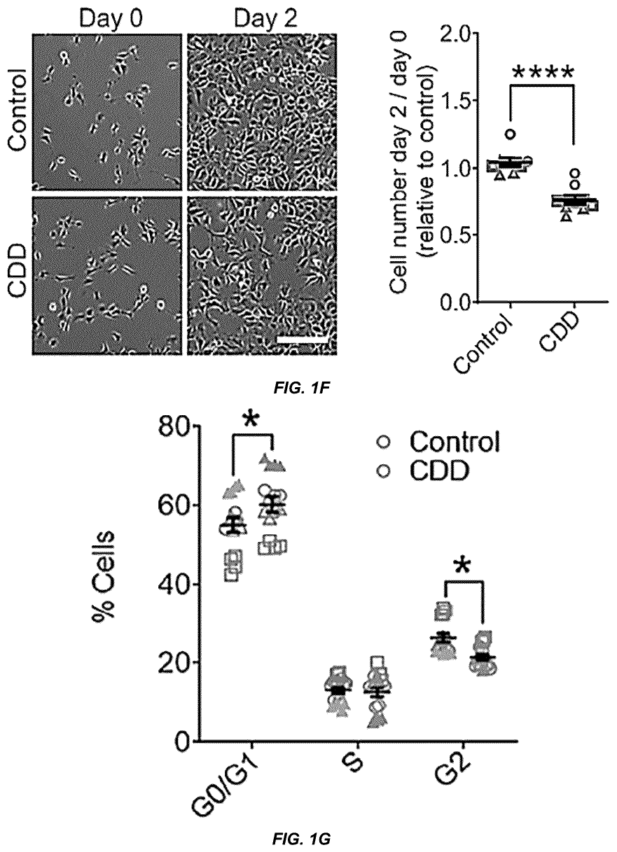

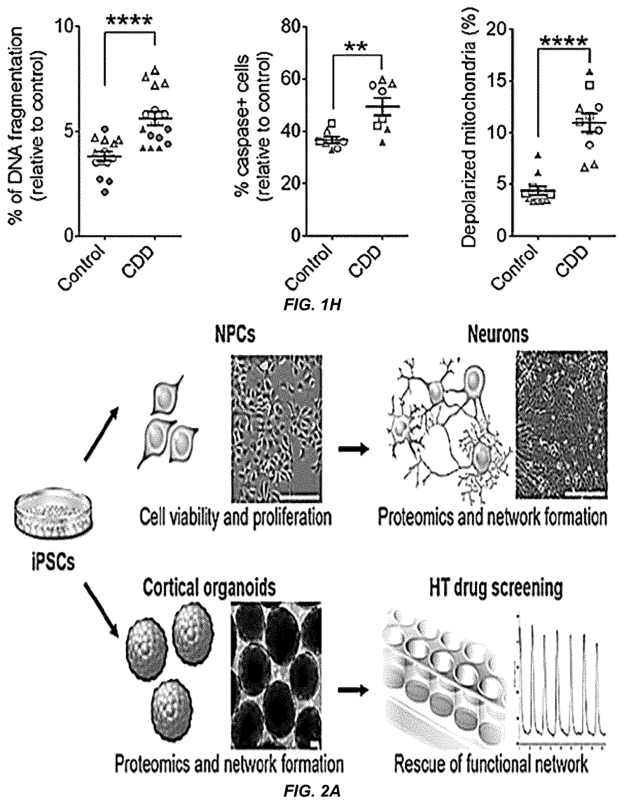

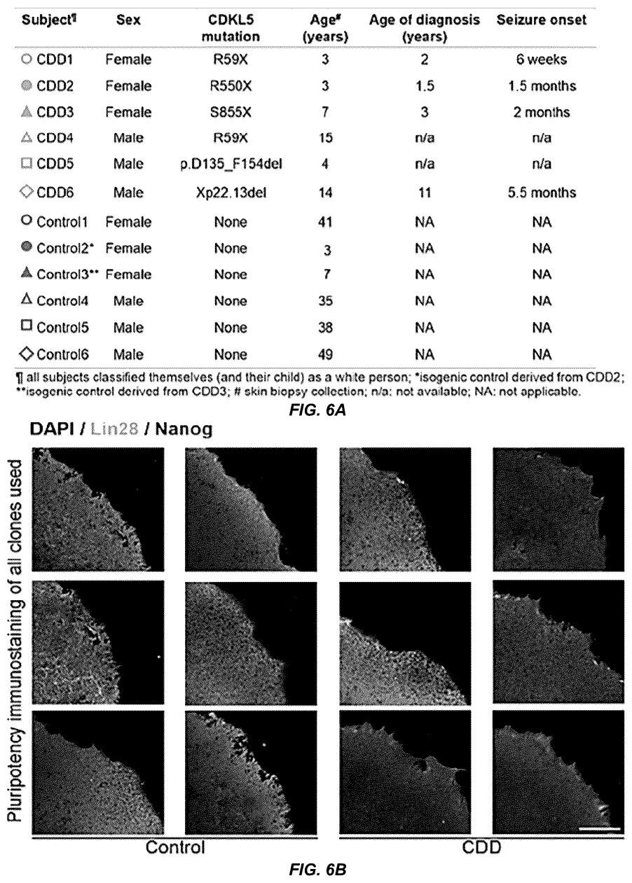

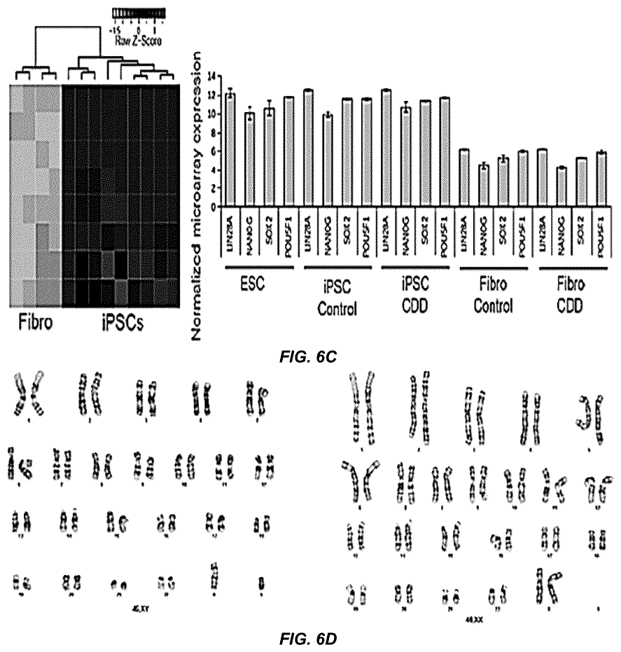

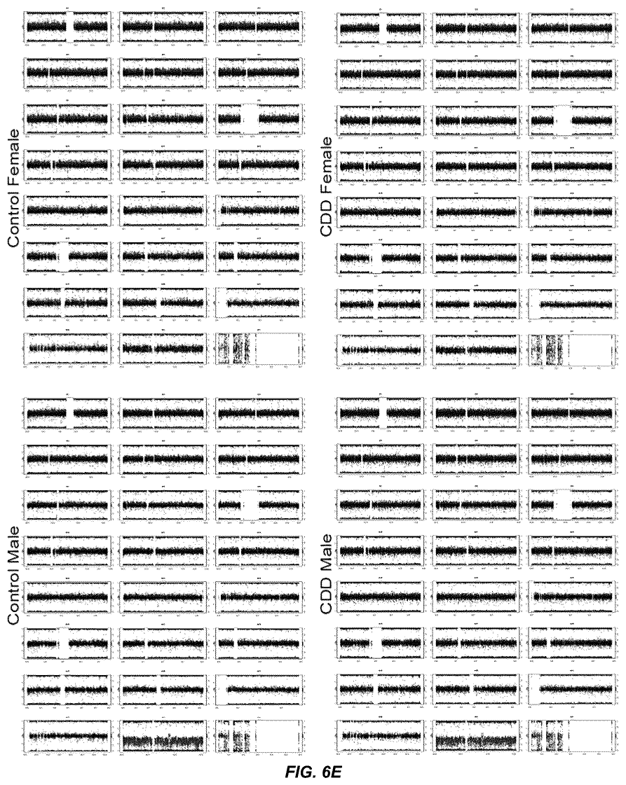

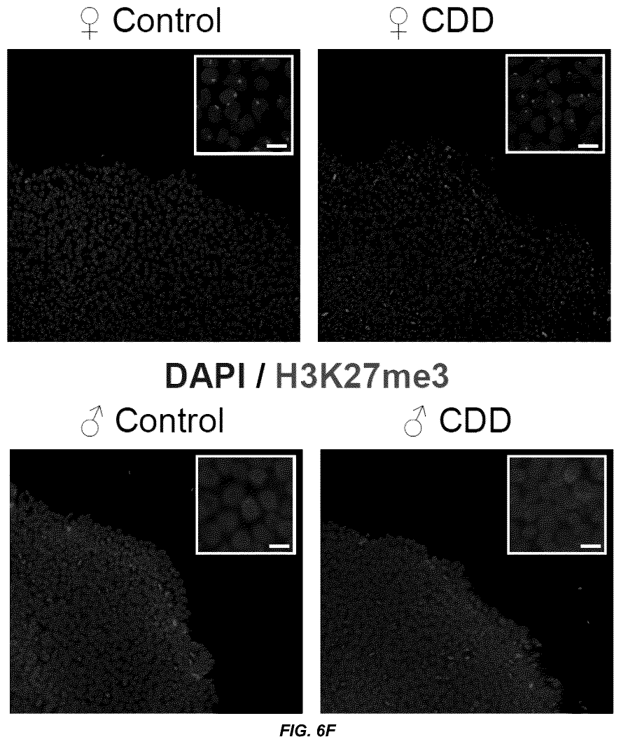

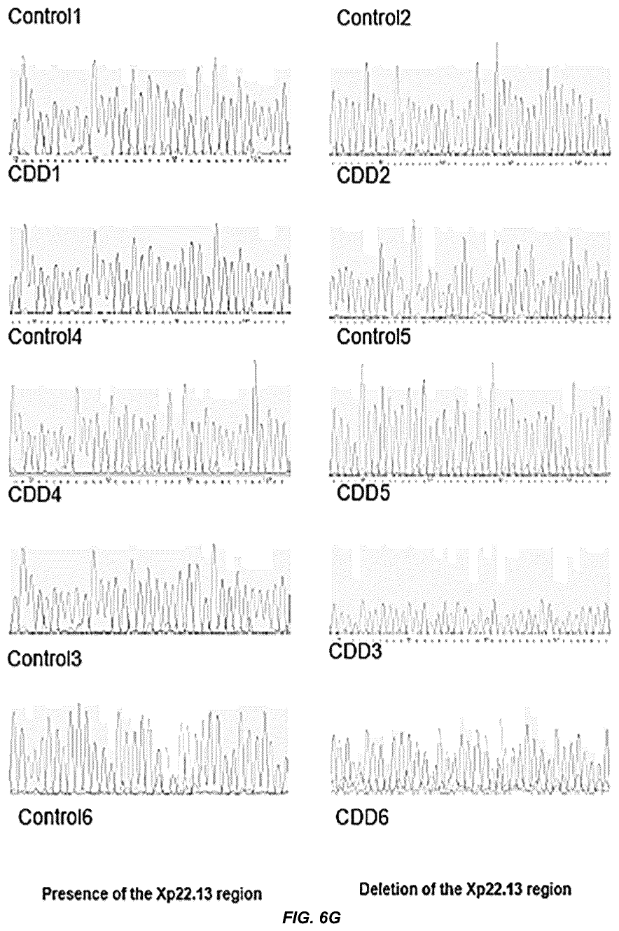

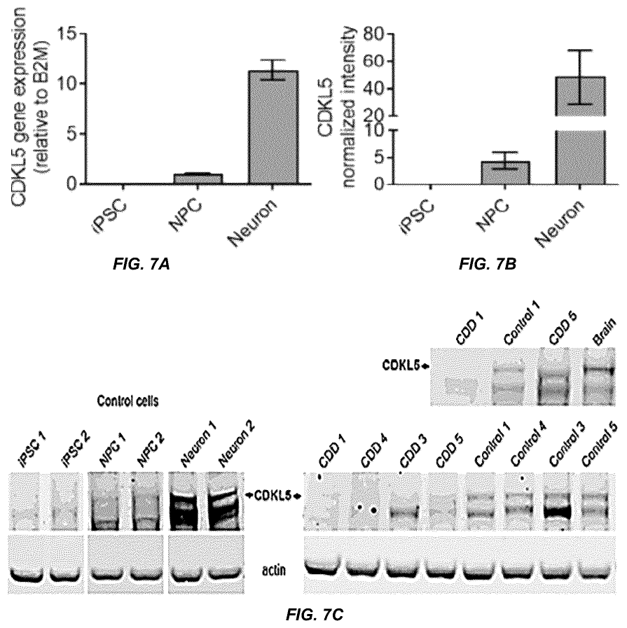

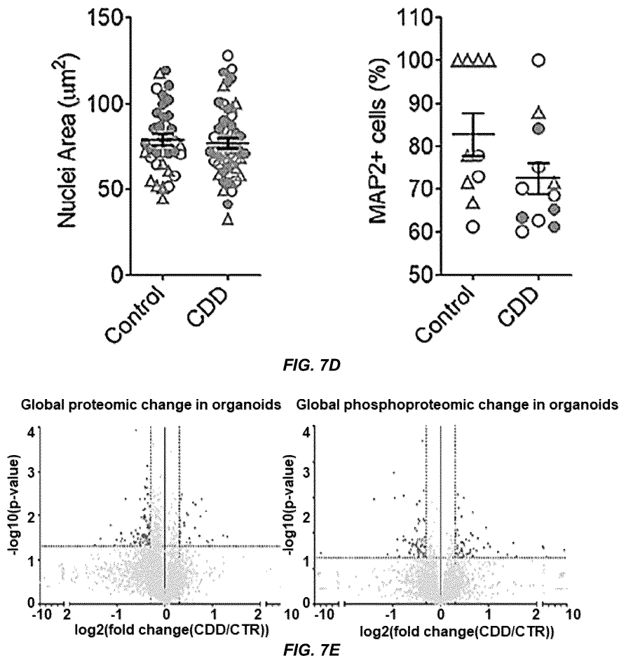

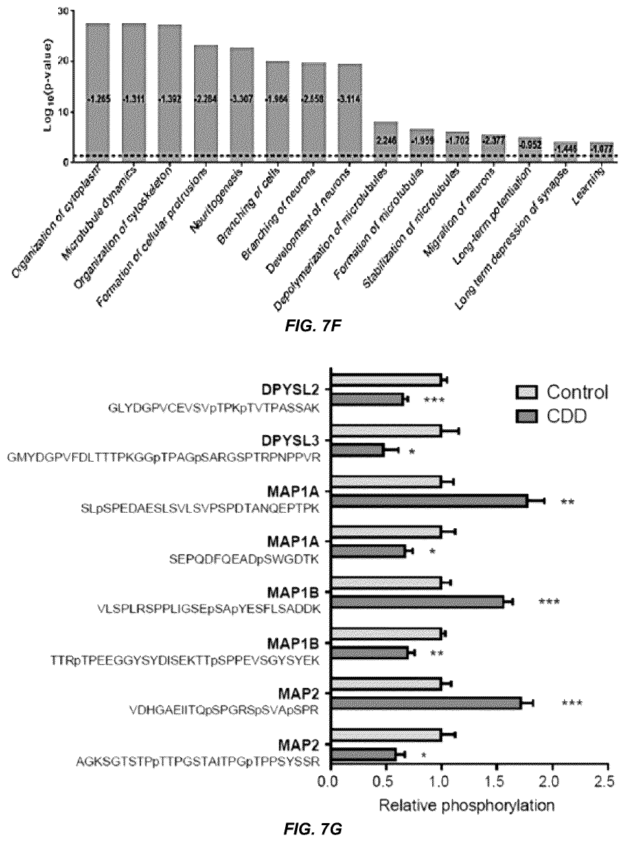

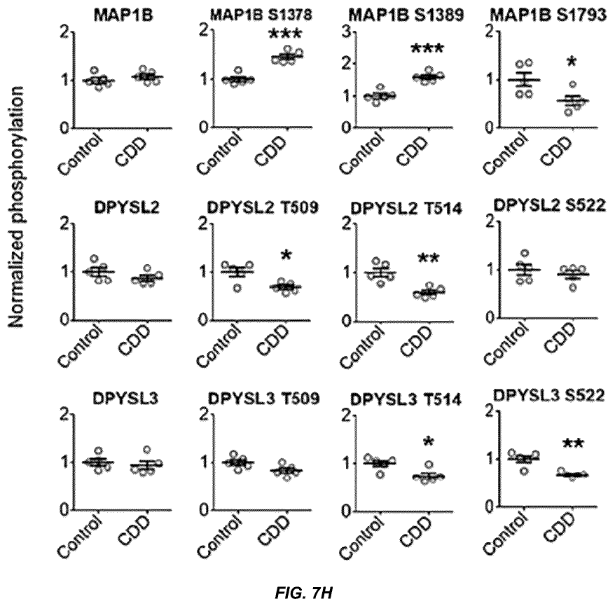

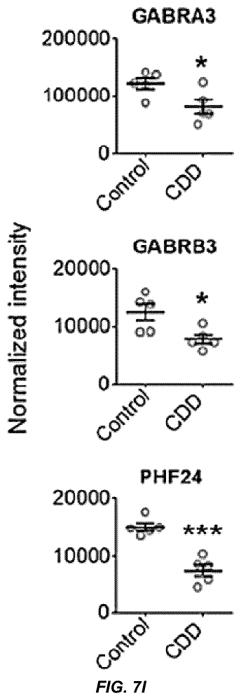

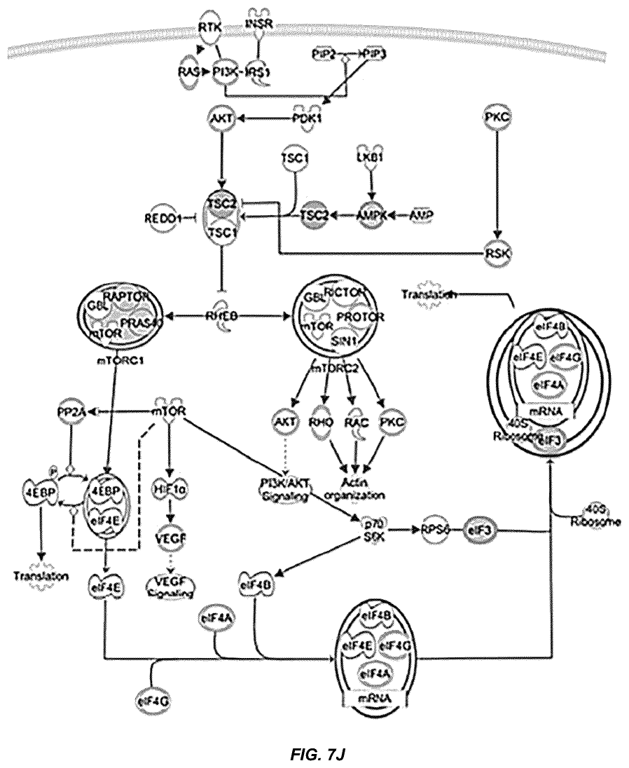

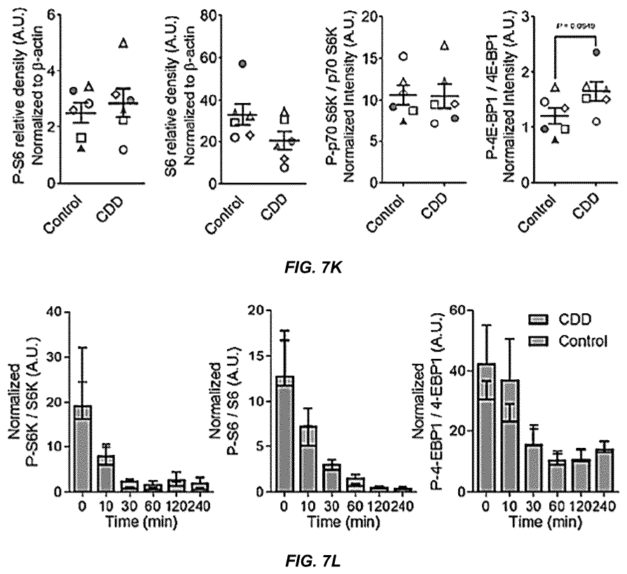

Proliferation defects and increased cell death in CDD neural progenitors. The impact of CDKL5 deficiency on human neurodevelopment was investigated after cellular reprogramming. Skin fibroblasts derived from six CDD patients, carrying five distinct CDKL5 mutations, were reprogrammed into iPSCs and fully characterized (FIGS. 1A and 6). All CDD iPSC lines do not express a functional CDKL5 protein (FIGS. 6 and 7A-C). This cohort includes additional iPSCs from sex-matched related controls, along with isogenic control clones for female subjects based on random X chromosome inactivation during cellular reprogramming (FIG. 6F-G). Each cell line is depicted with a coded symbol for easy visualization of the data on individual genotypes. A description of all experiments performed and cell lines used can be found in Table 2.

TABLE 2
Description of cell lines used

| Assay | CTL1 | CTL2 | CTL3 | CTL4 | CTL5 | CTL6 | CDD1 | CDD2 | CDD3 | CDD4 | CDD5 | CDD6 |
|---|---|---|---|---|---|---|---|---|---|---|---|---|
| CDKL5 gene sequencing | X | X | X | X | X | | X | X | X | X | X | |
| Microarray for Pluripotency | X | | | X | X | X | X | | | X | X | X |
| SNP-based Array for CNV (karyotyping) | X | X | X | X | X | X | X | X | X | X | X | X |
| G-banding Karyotyping | X | | X | | X | X | X | | X | | X | X |
| iPSC Staining | X | X | X | X | X | X | X | X | X | X | X | X |
| X Chromosome Inactivation Staining | X | X | X | X | | | X | X | X | X | | |
| NPC Staining | X | X | X | X | X | X | X | X | X | X | X | X |
| Neuronal Staining | X | X | X | X | X | X | X | X | X | X | X | X |
| NPC Proteomics and Phosphoproteomics | X | | X | X | X | | X | | X | X | X | |
| 2D Neurons Proteomics and P-proteomics | X | X | X | X | X | | X | X | X | X | X | |
| Organoid Proteomics and P-proteomics | X | X | X | X | | | X | X | X | X | | |
| Proliferation | X | | | X | X | | X | | | X | X | |
| Cell Cycle | X | | X | X | X | | X | | X | X | X | |
| DNA Fragmentation | X | X | X | X | | | X | X | X | X | | |
| Caspase Positive Cells | X | | X | X | X | | X | | X | X | X | |
| Mitochondrial Potential | X | | X | X | X | | X | | X | X | X | |
| CDKL5 Western Blot | X | X | X | X | X | X | X | X | X | X | X | X |
| PS6/S6 Western Blot | X | X | X | X | X | X | X | X | X | X | X | X |
| PS6K/S6K Western Blot | X | X | X | X | X | X | X | X | X | X | X | X |
| P4E-BP1/4E-BP1 Western Blot | X | X | X | X | X | X | X | X | X | X | X | X |
| Amino Acid Starvation | X | | | X | X | | X | | | X | X | |
| Nuclei Size Evaluation | X | | X | X | | | X | | X | X | | |
| MAP2 Positive Cell Counting | X | | X | | | | X | | X | X | | |
| Spine-like Motility | X | | | X | X | | X | | | X | X | |
| Spine-like Density | X | X | X | X | X | | X | | | X | X | |
| In vitro Neuronal Tracing & Sholl Analysis | X | X | X | X | X | | X | X | X | X | X | |
| In vivo Neuronal Tracing & Sholl Analysis | X | | | X | | | X | | | X | | |
| Cortical Organoid Size | X | | | X | | | X | | | X | | |
| Cellular Migration | X | X | | X | | | X | X | | X | | |
| Cellular Migration after Drug Treatment | X | | | X | X | | X | | | X | X | |
| Synapsin1 Western Blot | X | X | X | X | X | X | X | X | X | X | X | X |
| PSD95 Western Blot | X | X | X | X | X | X | X | X | X | X | X | X |
| Co-localized Puncta Quantification | X | X | X | X | X | | X | X | X | X | X | |
| Co-Immunoprecipitation (QMI) | X | | | X | | | X | | | X | | |
| Patch-clamping | X | | | X | | | X | X | | X | | |
| Multi-electrode Array (MEA) | X | | | X | X | | X | | | X | X | |
| High-Throughput Screening | X | | | | | | X | | | | | |

CTL = Control;
CDD = CDKL5 Deficiency Disorder

The iPSCs were differentiated into NPCs. Considering the CDKL5 kinase function, mass spectrometry-based quantitative proteomics and phosphoproteomics was performed to gain insights into its targets in a relevant cell type (FIG. 1B-E). 6505 proteins were identified, quantitated and differentially expressed protein (DEP) as proteins with t-test p-value<0.05 and log 2 (fold change) cutoff of >0.3 (12 proteins) or <−0.3 (124 proteins). The phosphoproteomics dataset, proteomic (nonphospho) analysis of CDD NPCs showed enrichment of microtubule-based process (23 proteins) among the down-regulated proteins (FDR q-value for overlap: 4.14E-4, 23 proteins). Many of these proteins (TUBGCP5, HAUS3, NDE1, NDC80, FBXO5, CUL7, ID2, KIF15, CHECK2) are also related to the mitotic cell cycle. Dysregulated phosphorylation or differential expression of proteins regulating cellular structures might hamper the proper neuronal development and plasticity. The DEP changes also pointed to an impairment in cell viability and neuronal development in CDD (FIG. 1D-E). Therefore, the proteomic signature pathways found in NPCs promoted further evaluate the proliferative aspect of these cells. By assessing in vitro cell number over time, CDD NPCs were observed to proliferate slowly compared to controls. Similar results of an altered cell cycle were obtained using image-based approaches (FIG. 1F-G). Additionally, increased DNA fragmentation, mitochondrial depolarization and caspase activity were observed (FIG. 1H). Together with an unaltered proportion of neurons following differentiation compared to control (FIG. 7d), these results suggest compromised proliferation and increased cell death in CDD neural cultures.

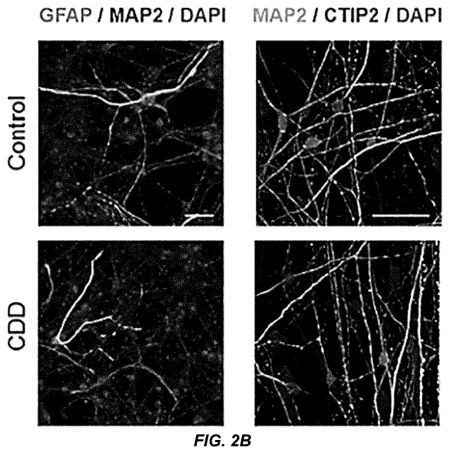

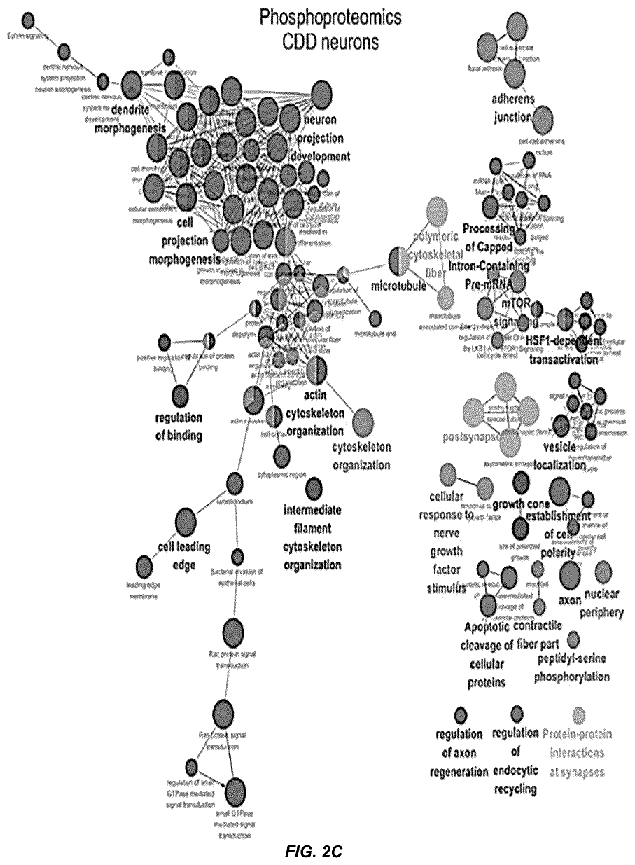

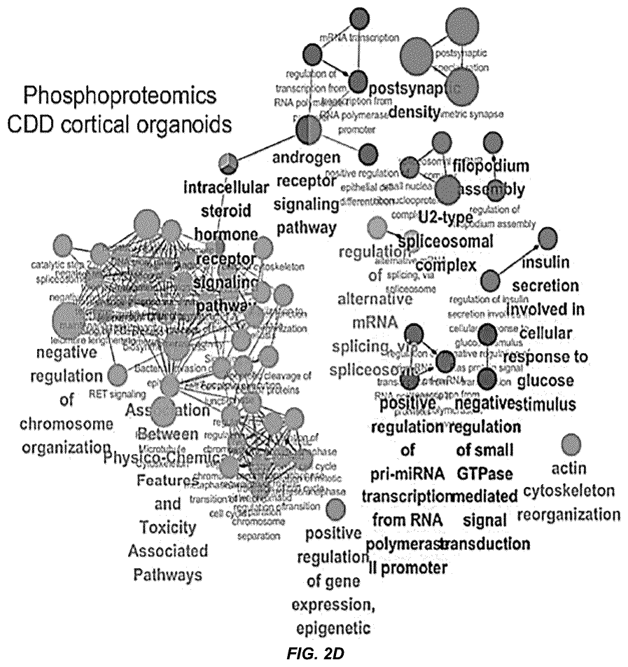

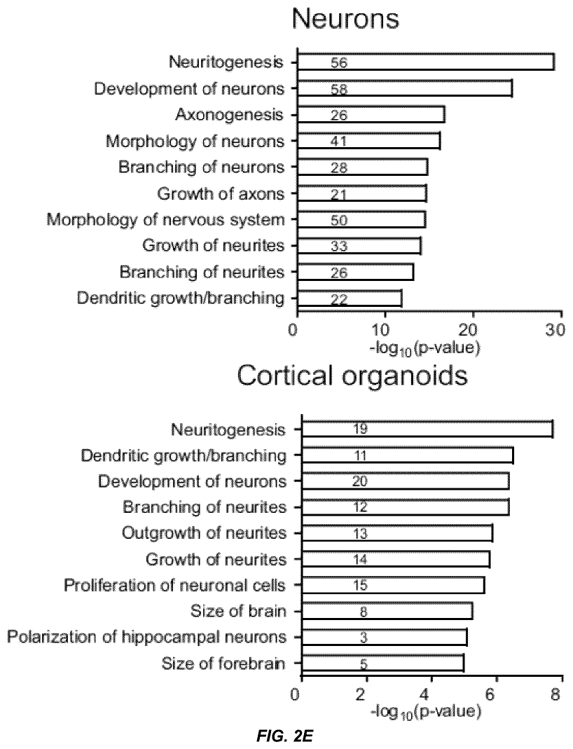

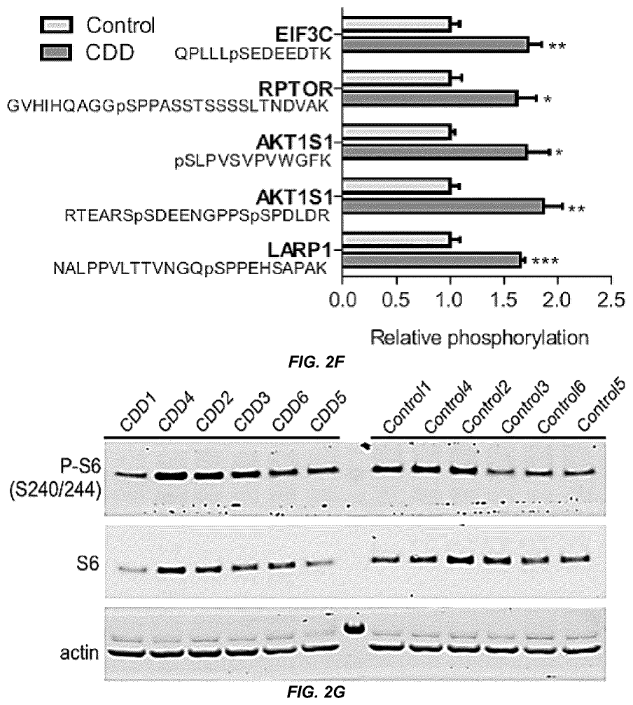

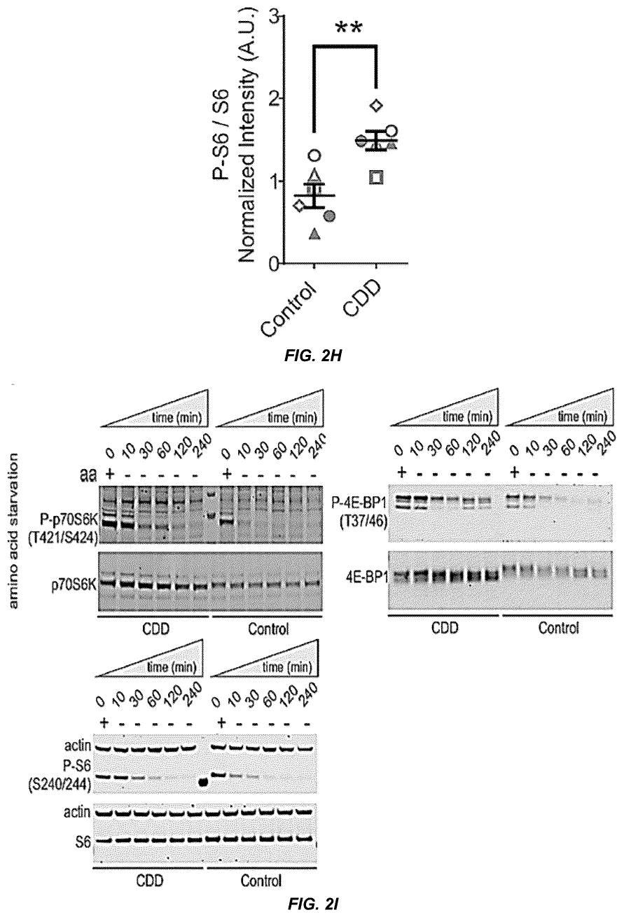

Phosphoproteomics analysis predicts alterations in the development and mTOR activation in CDD neurons. To further understand the impact of CDKL5 absence in the brain, cells were differentiated into cortical neurons and cortical organoids (FIG. 2A-B). The CDKL5 protein was not detected in iPSCs, mildly expressed in NPCs, and upregulated in neurons (FIG. 7A-C). The samples were analyzed by mass spectrometry following the phosphopeptide enrichment. In 2D 6-week-old neurons, 4115 phosphopeptides from 1485 proteins were identified and quantified. After normalization of the phosphopeptide intensity, the most significantly changed phosphopeptides were determined as those with |log 2 (fold change)|>0.5 and t-test p-value<0.05. In 2-month-old cortical organoids, 6769 phosphopeptides were identified and quantified from 2212 proteins (|log 2 (fold change)|>0.3 and t-test p-value<0.05, 120 phosphopeptides from 158 proteins, FDR≤0.05).

Motif analyses detected significantly enriched sequences around the phosphosites, with over-representation of pSXXE among the up-regulated phosphopeptides, which is phosphorylated by acidophilic kinases such as CK2. Ingenuity Pathway Analysis (IPA) and Kinase Enrichment Analysis (KEA) revealed that phosphoproteins known to be phosphorylated by CK2 were increased in CDD neurons, suggesting that CK2 was over-activated. Over-representation of pSP motif was also found among the down-regulated phosphopeptides in CDD neurons. CDKL5 was not detected as an upstream kinase regulator, presumably due to the limited knowledge about its substrates. Although down-regulation of phosphoproteins might be linked with inhibition of other proline-directed kinases, a subset of the down-regulated phosphoproteins may be the direct substrates of CDKL5.

The phosphoproteomic analysis revealed several dysregulated phosphoproteins in CDD neurons and organoids (FIG. 2D-G and FIG. 7E-K). Altered phosphorylation of microtubule-associated proteins such as MAP1A, MAP1B, MAP2 and collapsin response mediator protein (CRMP) family proteins (DPYSL2 and DPYSL3) were also found in these cell types; along with cytoskeleton organization proteins, such as CTTN. The overall enriched terms in neurons and organoids include neuritogenesis, morphology, migration, synaptogenesis, and neurotransmission (FIG. 2F; FIG. 7F).

Among the down-regulated proteins observed in the proteomic analysis, the gamma-aminobutyric acid (GABA) signaling pathway was also over-represented (FIG. 7I). For the up-regulated proteins, markedly enriched GO terms were lysosome, glycosphingolipid metabolic process, and aminoglycan catabolic process. Up-regulated proteins such as GALNS, GM2A, EPHA2, RHOG, HEXA, and HEXB are involved in the degradation of chondroitin sulfate, a sulfated glycosaminoglycan that participates in neurite outgrowth. Moreover, in the phosphoproteomic analyses of cortical neurons, mTOR pathway-related phosphoproteins were enriched (FIG. 2G and FIG. 7J). Increased phosphorylation of proteins such as AKT1S1 (also known as PRAS40), EIF3C, RPTOR, and LARP1, suggested that the pathway is activated in CDD cells. Thus, experiments were performed that confirmed the increased phosphorylation of ribosomal subunit S6 protein levels in CDD neurons (FIG. 2H-I and FIG. 7K).

To further validate these results, experiments were performed to test whether CDD neural cells are impaired in their ability to turn off mTORC1 downstream proteins upon amino acid starvation. When cultured with medium lacking amino acids, control cells shut off mTORC1 activity faster and to lower levels than CDD cells. In contrast, CDD cells are significantly impaired in their response to amino acid removal, retaining mTORC1 activity levels for longer. Unlike control cells, in which the phosphorylation levels of p70S6K, S6 and 4E-BP1 are almost inexistent after 60 min of amino acid withdrawal, CDD cells have a delayed response exhibiting only a partial drop (FIG. 2J and FIG. 7L). Therefore, in addition to alterations in cytoskeleton organization and neurotransmission, the mTOR pathway activation predicted by proteomic and phosphoproteomic signatures of CDD neurons was confirmed by amino acid starvation in CDD cells.

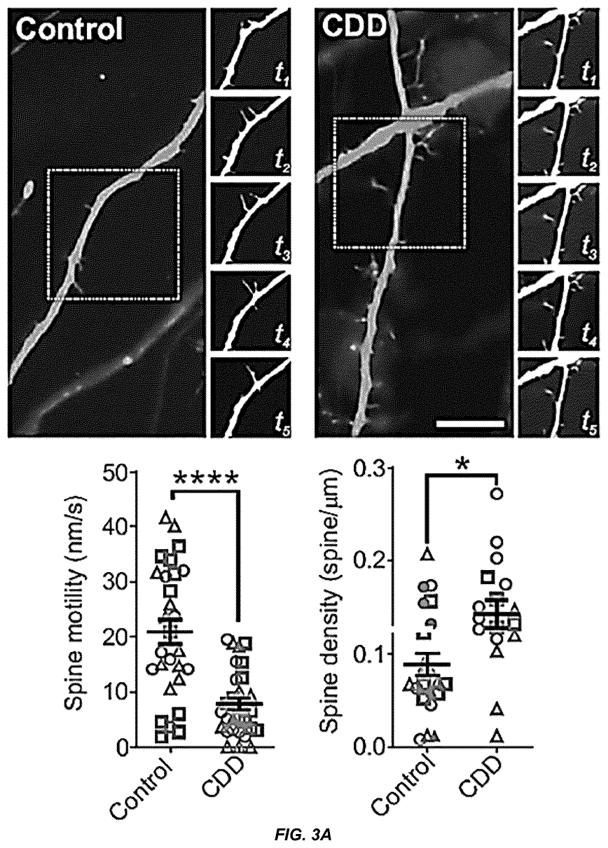

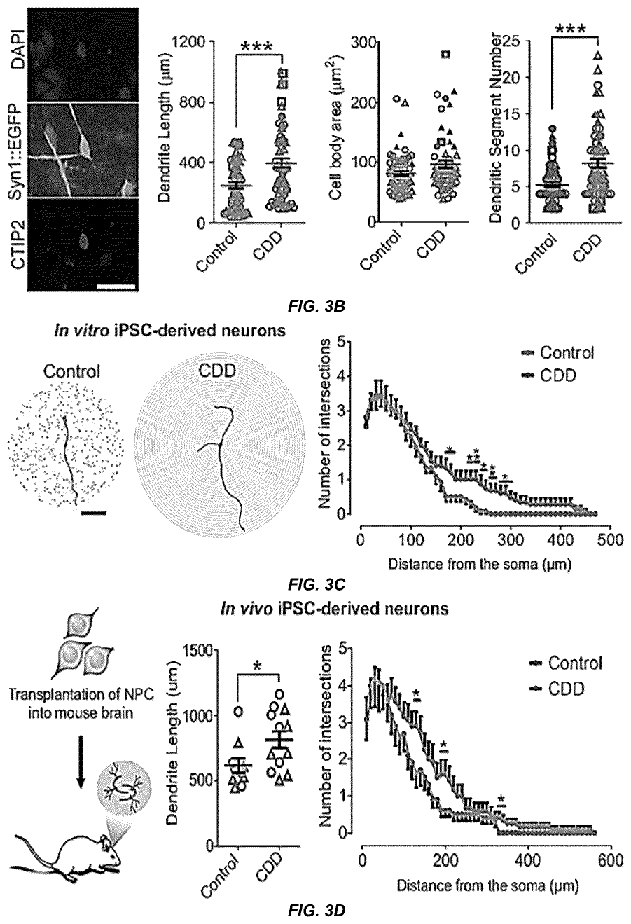

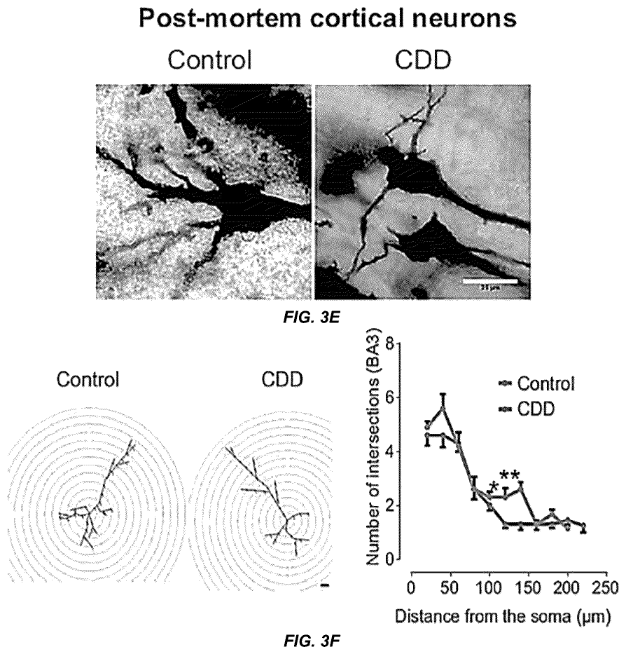

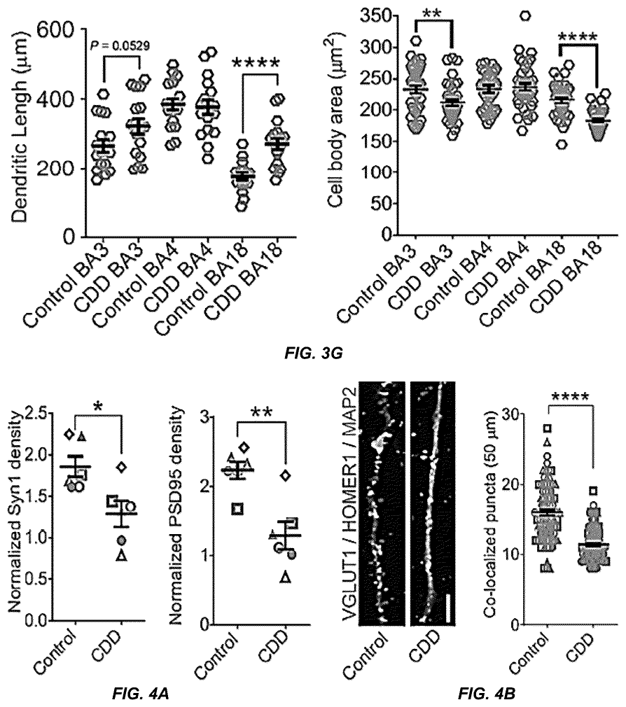

CDD neurons display morphological abnormalities and impaired migration. Cytoskeletal proteins structurally support the neuronal dendrites and axons, and play essential roles in neuronal morphogenesis, plasticity, and migration. Since cytoskeletal protein alterations were highlighted by the proteomics, measurements of neurite outgrowth and spine-like dynamics were performed. Neurons stably transfected to express EGFP by Synapsin1 promoter self-inactivating lentivirus were dissociated, re-platted, and had their neurites recorded over time. It was observed that the spine-like motility was significantly reduced, while spine-like density was increased in CDD neurons (FIG. 3A). These cellular observations agreed with the alterations detected on microtubule dynamics and cytoskeleton organization.

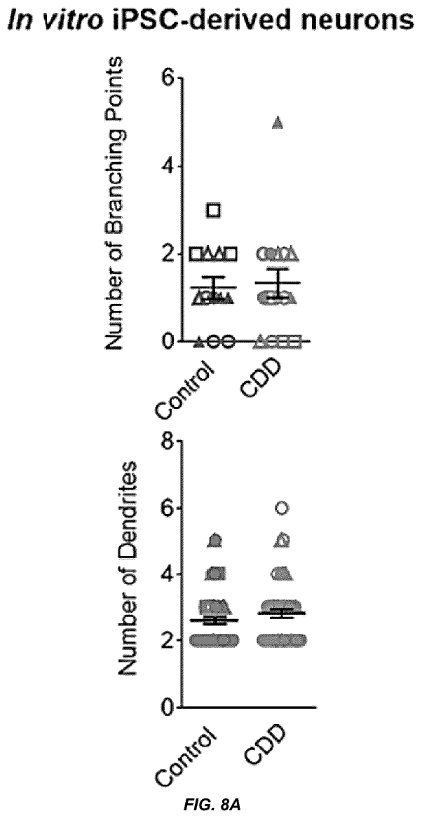

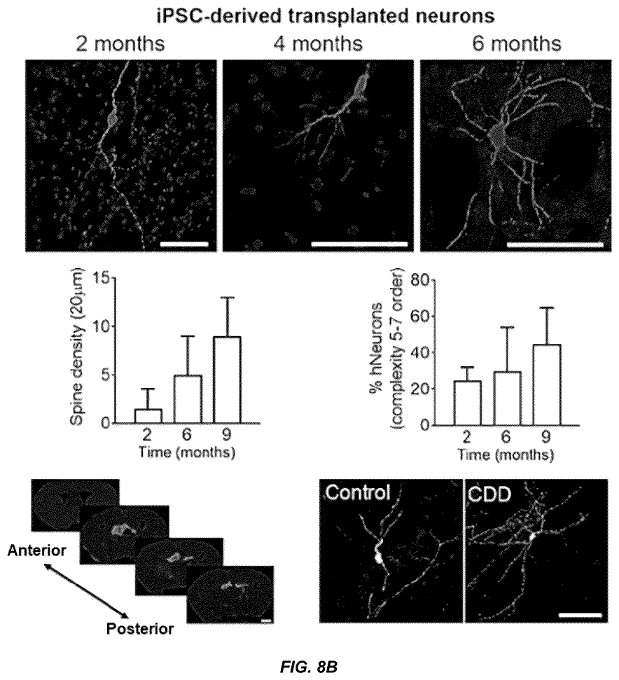

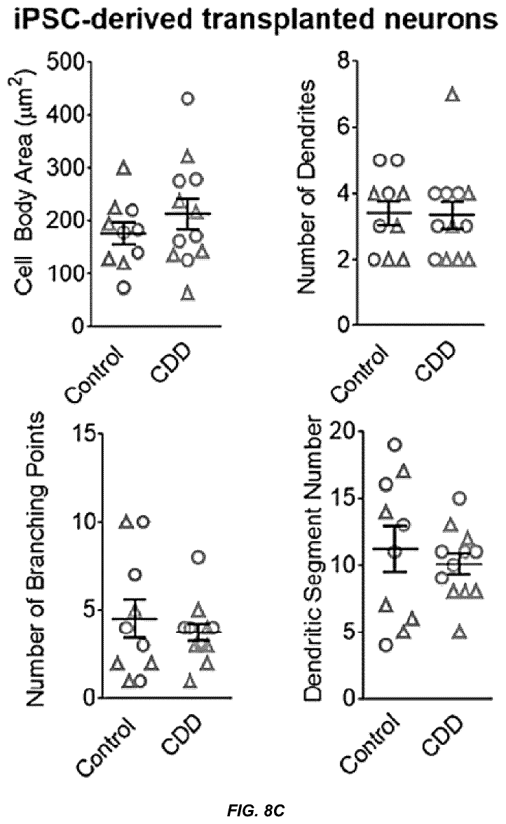

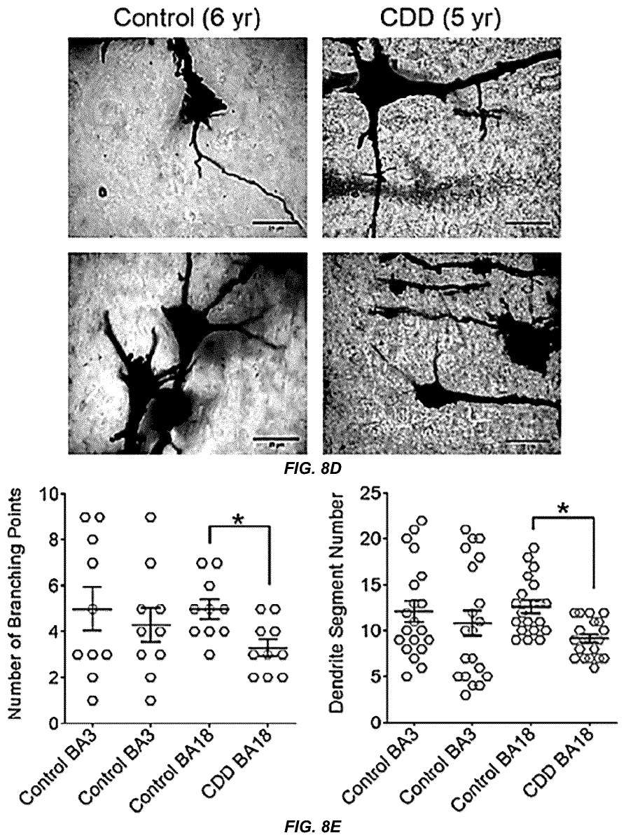

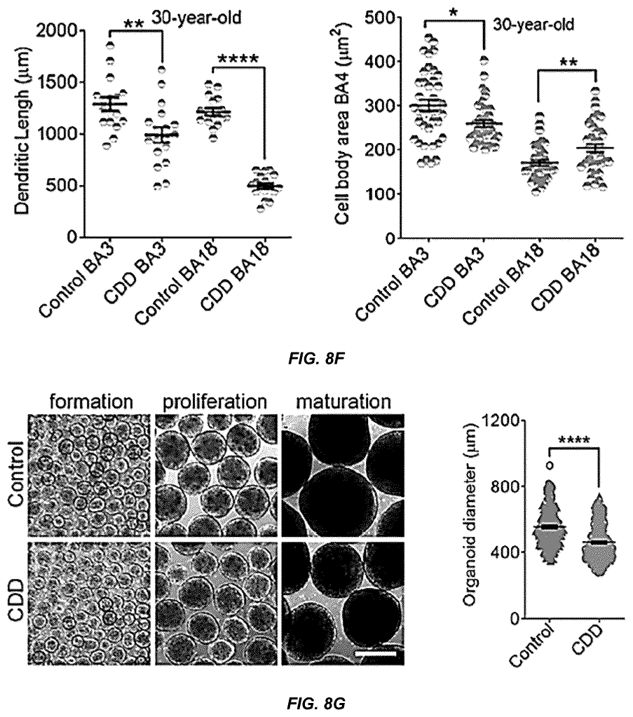

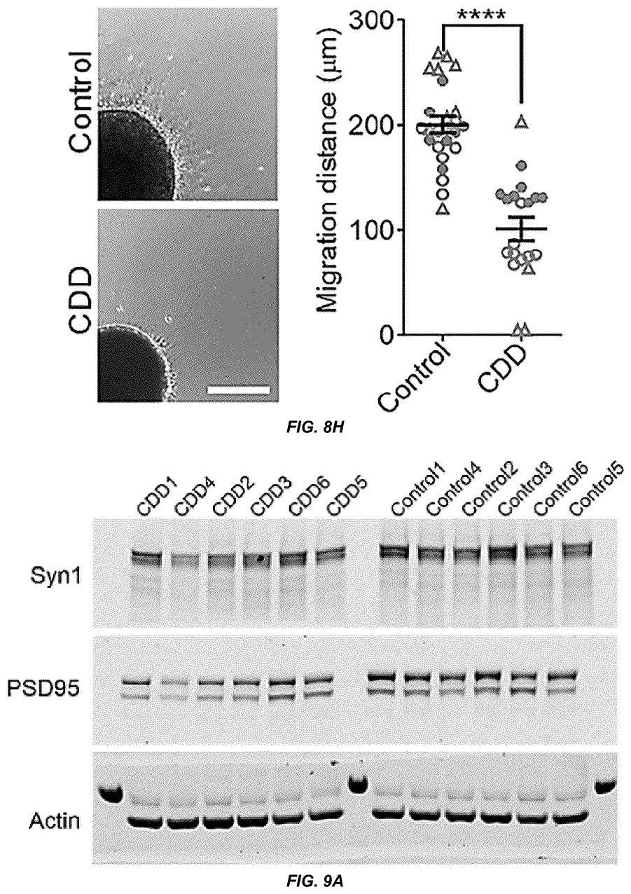

The total dendritic length and complexity of CDD neurons were significantly higher than controls (FIG. 3B-C and FIG. BA). To determine whether these phenotypes were cell autonomous, human NPCs, previously transduced with SYN1:EGFP to label neurons, were transplanted into newborn mouse brains. Since the brain environment promotes a faster neuronal maturation of human transplanted cells, this strategy was used to highlight the cell-autonomous phenotypes of CDD neurons in a more pronounced fashion for extended periods (FIG. 3D and FIG. 8B). Ten to twenty newborn mice were transplanted, and a fraction of the human cells integrates individually or in small clusters into the host brain with similar morphometric dimensions as adjacent host cells. Six months after grafting, the transplanted cells were integrated and differentiated in the mouse brain. The EGFP-positive human neurons were 3D reconstructed, and the morphological features were analyzed. A significant increase in dendritic length was found in transplanted CDD neurons, in agreement with the in vitro CDD morphological alterations (FIG. 3D and FIG. 8C).

In an attempt to place the findings in the broader context of the cortical morphology in human participants at the cellular level, orthogonal validation experiments were conducted using postmortem brain tissues from gender-, age- and hemisphere-matched CDD and control subjects (FIG. 3E-G and FIG. BD-F). Considering the limited availability of samples, the human tissue was used to test predictions based on the neuronal differences found in vitro, validating possible alterations in the morphology of cortical neurons. There was heterogeneity of results in vivo depending on the specific cortical area under investigation (Broadman area, BA) and, most importantly, on the individual's age. Similar to the in vitro data, BA3 and BA18 cortical neurons in postmortem tissue from a young CDD patient (5-year-old) displayed larger total dendritic length (FIG. 3G). However, these differences change in neurons obtained from an older patient (30-year-old. FIG. SF), indicating that the development stage in which CDD neuronal morphology is assessed is crucial for comparisons. Additionally, it was observed that CDD proliferation and migration alterations lead to smaller cortical organoids and migration defects compared to controls (FIG. BG-H).

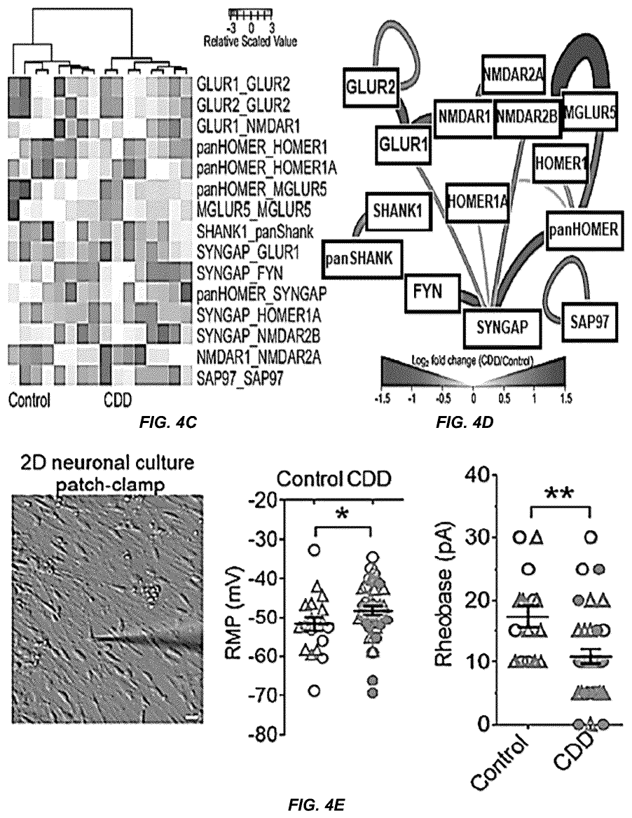

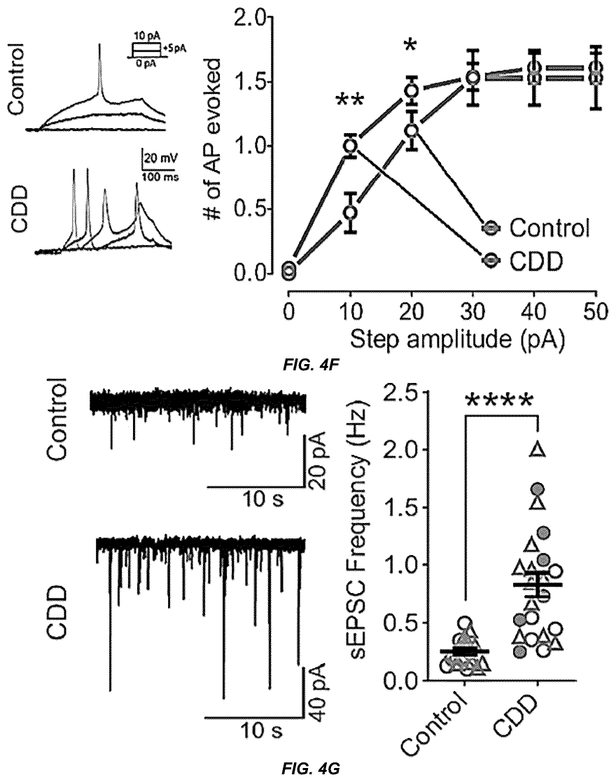

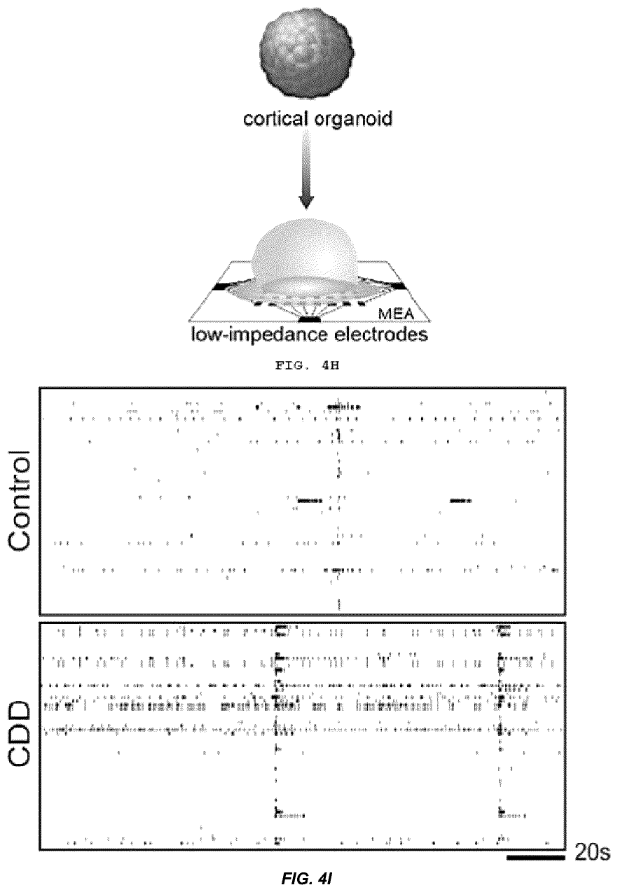

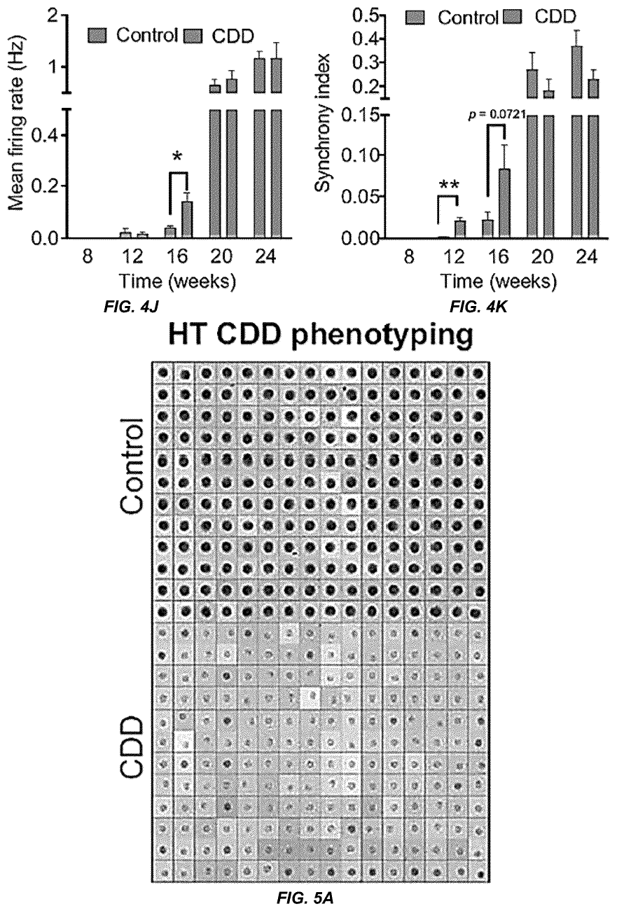

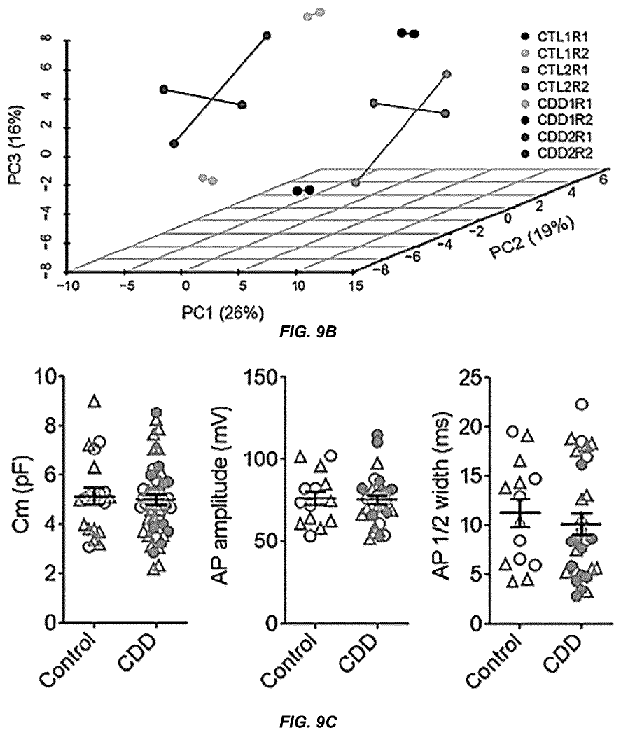

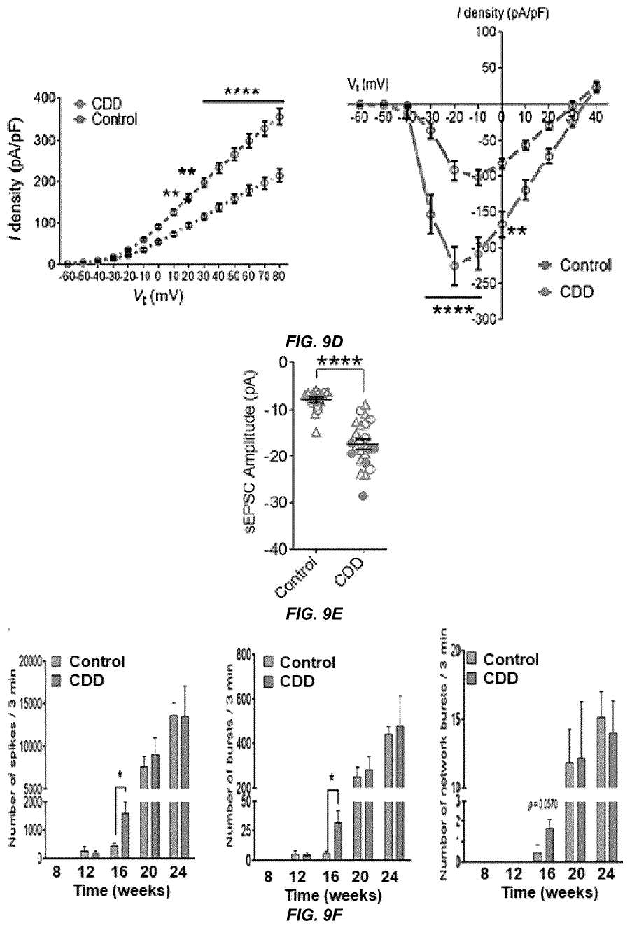

CDKL5 deficiency impacts synaptogenesis and promotes early network hyperexcitability. Next, experiments were performed to investigate how the cellular and molecular alterations found in CDD would impact neuronal functionality and network formation. Decreased expression of pre- and post-synaptic protein markers (Syn1 and PSD95, respectively) was observed by Western blot (FIG. 4A and FIG. 9A), and independently confirmed by the reduced density of co-localized VGLUT1 and HOMER1 synaptic puncta on MAP2-positive processes of CDD neurons (FIG. 4B).

To determined how CDKL5 deficiency might affect the dynamic protein co-associations of glutamatergic synapse-associated proteins, quantitative multiplex co-immunoprecipitation (QMI) was used to measure the abundance of protein co-associations among a set of 22 synaptic proteins. The protein content was analyzed from cortical organoids derived from two CDD and two related control individuals in duplicate, two separate times to quantify the effect of batch, genetic background, and genotype. Principal component analysis and hierarchical clustering revealed a main effect of CDKL5 deficiency, with apparent clustering of CDD compared to controls. Within these major groups, technical replicates differentiated in parallel clustered more closely than genetically identical lines, suggesting moderate batch effects (FIG. 9B). Nonetheless, 15 protein co-associations were significantly different between control and CDD carriers (FIG. 4C-D). The amount of immunoprecipitated mGluR5_probe mGluR5, and mGluR5_Homer was strongly upregulated, as was the amount of GluR1_GluR1 and GluR1_GluR2, indicating altered composition and scaffolding of synaptic glutamate receptors. Lower levels of many interactions involving SynGAP were also detected, indicating reduced incorporation of this activity-dependent signaling protein into postsynaptic receptor complexes. These data demonstrate widespread changes in synaptic protein networks downstream of CDD neurons with the possible implication in the neural network formation.

To determine the impact of disrupted glutamatergic signaling on developing neuronal networks, experiments started at a single-cell level and performed a whole-cell patch-clamp recording of iPSC-derived 2D monolayer cortical neurons (10-week-old neurons). While the resting membrane potential (RMP) was increased, a significantly lower rheobase current was observed in CDD neurons (FIG. 4E). Rheobase is a measure of membrane potential, indicating the minimal current amplitude that results in action potential (AP). No changes were observed in the membrane capacitance, AP amplitude and width (FIG. 9C). The higher number of APs evoked by +10, and +20 pA current injections and a lower rheobase suggest higher neuronal excitability in CDD neurons compared to controls (FIG. 4F). In addition, the CDD neurons exhibited significant increases in Na⁺ current (I_{Na}) and transient A-type current (I_{KA}) densities, and a negative shift of channel activation compared with their corresponding controls, supporting the hyperexcitability profile (FIG. 9D). Corroborating these findings, increased sEPSC frequency and decreased amplitude were observed in CDD cortical neurons (FIG. 4G and FIG. 9E). Together, this revealed the intrinsic hyperexcitability and dysfunction of voltage-gated Na+ and K+ channels that underlie the electrical hyperactivity in CDD cortical neurons.

To further evaluate the cortical organoid functionality at a mesoscopic level, weekly extracellular recordings were obtained of spontaneous electrical activity using multi-electrode arrays (MEA). Around four cortical organoids were plated per well in a MEA plate containing 64 microelectrodes with 30 μm of diameter spaced by 200 μm (FIG. 4H). The spikes were defined by the event unit waveforms standard structure with typical refractory periods. For approximately 6 months, cortical organoids exhibited consistent increases in electrical activity, as parametrized by channel-wise firing rate, burst frequency, and synchrony. Furthermore, a significant increase in spike frequency and an overly synchronized network at the early stages of development was observed in CDD neuronal networks (FIG. 4I-K and FIG. 4F). Altogether, these results highlight the contribution of a specific gene in the emergence of a hyperexcitable and overly synchronized network.

High throughput drug screening and functional rescue of CDD neural network. After identifying the hyperexcitability of CDD neural networks, experiments were performed to determine if such phenotype could be pharmacologically reverted. For this purpose, a high throughput (HT) drug screening platform was developed composed of 3D neural spheroids in a 384-well format (one spheroid per well; FIG. 5 and FIG. 10). This platform was optimized to reduce variability within the genotypes, with spheroids displaying the same size across the plate, which is critical for a consistent and reproducible screening assay (FIG. 5A). Each spheroid contains a balanced population of cortical neurons and astrocytes expressing key markers of maturity and exhibits robust spontaneous, synchronous calcium oscillations. Although both spheroid genotypes showed an increase in neural activity over time, the frequency of spontaneous calcium oscillations (number of peaks) was more pronounced in CDD compared to controls, starting at early stages of the development; while the amplitude (peak height) was decreased in CDD neurons (FIG. 5B-C and FIG. 10E). In this context, calcium oscillations was selected as a physiological readout to avoid disrupting the established 3D network, while evaluating the spheroid size and viability.

Next, a unique library of 1112 neuronal signaling compounds was screened by chronically treating 3-week-old CDD spheroids in triplicate at 1 μM for 3 weeks (FIG. 5D; FIG. 10). The calcium peaks' baseline features were determined solely in the presence of each vehicle (DMSO or water) to avoid any confounding effect on the oscillatory activity (FIG. 5E and FIG. 10A-D). Similarly, the result from each compound was normalized against its vehicle control (DMSO or water). To detect potential toxic effects of specific drugs, spheroid size and cell viability were monitored (FIG. 5F-G).

The baseline functional signature of CDD spontaneous calcium oscillations compared to control was used to identify chemicals capable of rescuing such a hypersynchronous phenotype (FIG. 5H). To enable unbiased quantitative analysis of the impact of each treatment on all 17 functional parameters analyzed (including Calcium peak height, peak count and synchronization) an aggregate measure of overall system perturbation was used—Parameter Recovery (PR) and Scalar Perturbation (SP). Briefly, Parameter Recovery indicates the proportion of parameters rescued by each compound replicates by dividing the sum of the recovered parameters by the total number of parameters (100% indicates all parameters are recovered to control boundaries). Scalar Perturbation allows for estimating whether a given compound changes the recovery status by an adaptive response. The SP is essentially zero in untreated cells, but as the functional endpoint changes in response to the compound treatment, the SP increases. In other words, the SP and PR allowed for categorization of screened compounds: the smaller or higher the score, respectively for SP or PR, the closer the CDD calcium oscillatory activity is from control (the top lead compounds are highlighted. FIG. 5I-J). By investigating the correlation between the top 50 compounds, enriched terms included neuronal signaling, protein tyrosine kinase, MAPK, cell cycle and PI3K/Akt/mTOR (FIG. 10H). These findings support the validity of this platform and prompted selection of the top lead.

After combining the analysis of the multi-parametric pipeline, four chemicals were identified that demonstrated strong therapeutic potential by rescuing CDD's altered functional phenotype in more than 60% with no effect on cell viability: the hyperpolarization-activated cyclic nucleotide-gated (HCN) channel blocker Ivabradine (SP ranking=10, PR ranking=3,); the muscarinic receptor inhibitor Solifenacin (SP ranking=28, PR ranking=4); the potent and selective GSK3 inhibitor AZD1080 (SP ranking=12, PR ranking=23), and the Notch inhibitor Crenigacestat (SP ranking=5, PR ranking=10) (FIG. 5K-L). Further experiments were performed to determine if these compounds could display a dose-dependence action in a similar approach, where the CDD spheres were treated with the top 4 compounds in 6 different concentrations (FIG. 5M). Using a nonlinear regression fit, Ivabradine exhibited EC₅₀=0.041 μM with HillSlope=1.00 and Crenigacestat, EC₅₀=0.883 μM with HillSlope=39.40.

Next, these lead compounds were investigated for their ability to ameliorate CDD cellular phenotype. Following a similar pharmacological strategy, spheres were treated for 3 weeks, and cellular migration was evaluated after plating. Ivabradine, Solifenacin, AZD1080, and Crenigacestat were able to rescue outward radial cellular migration defects in CDD. Altogether, the platform revealed novel compounds that could be therapeutically useful for CDD and other types of refractory epileptic syndromes.

It will be understood that various modifications may be made without departing from the spirit and scope of this disclosure. Accordingly, other embodiments are within the scope of the following claims.

1. A method for treating a disease or disorder caused by abnormal CDKL5 expression in a subject in need thereof, comprising administering to the subject a therapeutically effective amount(s) of an agent selected from the group consisting of Ivabradine, Solifenacin, AZD1080, Crenigacestat and any combination thereof.

2. The method of claim 1, wherein a single pharmaceutical composition formulated for oral delivery comprises the Ivabradine, Solifenacin, AZD1080, Crenigacestat or any combinations thereof.

3. The method of claim 1, wherein the abnormal CDKL5 expression is cause by a genetic disease or disorder that affects the expression of the CDKL5 gene or the activity of the CDKL5 protein.

4. The method of claim 3, wherein the genetic disease or disorder is CDKL5 deficiency disorder (CDD).

5. The method of claim 1, wherein the subject is a female subject.

6. The method of claim 1, wherein the subject is a male subject.

7. The method of claim 1, wherein the subject is less than 25 years of age.

8. The method of claim 7, wherein the subject is less than 10 years of age.

9. A method of treating CDKL5 deficiency disorder (CDD) in a subject, comprising administering to the subject a therapeutically effective amount(s) of an agent selected from the group consisting of Ivabradine, Solifenacin, AZD1080, Crenigacestat and any combination thereof.

10. The method of claim 9, wherein a single pharmaceutical composition formulated for oral delivery comprises the Ivabradine, Solifenacin, AZD1080, Crenigacestat or any combinations thereof.

11. The method of claim 9, wherein the CDD is the result of abnormal CDKL5 expression.

12. The method of claim 11, wherein the abnormal CDKL5 expression is cause by a genetic disease or disorder that affects the expression of the CDKL5 gene or the activity of the CDKL5 protein.

13. The method of claim 9, wherein the subject is a female subject.

14. The method of claim 9, wherein the subject is a male subject.

15. The method of claim 9, wherein the subject is less than 25 years of age.

16. The method of claim 15, wherein the subject is less than 10 years of age.

17. A pharmaceutical composition comprising at least 2 agents selected from the group consisting of Ivabradine, Solifenacin, AZD1080, Crenigacestat and salts of any of the foregoing.

18. A human-based neural drug screening platform for identifying therapeutic compounds that can ameliorate or rescue deleterious biological effect(s) resulting from abnormal expression or activity of CDKL5, comprising:
(a) contacting set(s) of neurons, that have been differentiated from human stem cells, with a candidate drug, wherein a first set of neurons have been differentiated from human pluripotent stem cells that have mutation(s) affecting the normal expression of a CDKL5 gene and optionally, a second set of neurons that are differentiated from pluripotent stem cells that do not have said mutation(s); and/or
(b) contacting set(s) of neurospheres, that have been generated from human stem cells, with the candidate drug, wherein a first set of neurospheres comprises neurospheres that have been differentiated from human pluripotent stem cells that have mutation(s) affecting the normal expression of a CDKL5 gene, and optionally, a second set of neurospheres that have been differentiated from the human pluripotent stem cells that do not have said mutation(s); and/or
(c) contacting set(s) of cortical organoids, that have been generated from human stem cells, with the candidate drug, wherein a first set of cortical organoids have been differentiated from human pluripotent stem cells that have mutation(s) affecting the normal expression of a CDKL5 gene, and optionally, a second set of cortical organoids that have been differentiated from the human pluripotent stem cells that do not have said mutation(s);
(d) evaluating whether the candidate drug rescues or ameliorates deleterious biological effect(s) resulting from abnormal expression of a CDKL5 gene in the first set of neurons, and/or the first set of neurospheres, and/or the first set of cortical organoids,
wherein if a candidate drug rescues and/or ameliorates one or more deleterious biological effects the candidate drug is a therapeutic compound.

19. The platform of claim 18, wherein the platform comprises neurons, neurospheres and/or cortical organoids that have been produced from induced pluripotent stems cells.

20. The platform of claim 19, wherein the induced pluripotent stem cells are dedifferentiated from cells isolated from a subject having a CDKL5 deficiency disorder (CDD).

21. (canceled)
